(19) United States
(12) Patent Application Publication
(10) Pub. No.: US 2021/0383915 A1
(43) Pub. Date: Dec. 9, 2021

(54) SYSTEMS AND METHODS FOR PROCESSING ELECTRONIC IMAGES TO DETERMINE A MODIFIED ELECTRONIC IMAGE FOR BREAST PROCEDURES

(71) Applicants: Gerardo MORA, Alajuela (CR); Gerson RODRIGUEZ, Alajuela (CR); Raghu RAGHAVAN, Baltimore, MD (US); Martin BRADY, Baltimore, MD (US); Roberto DE MEZERVILLE, Alajuela (CR); Juan CHACÓN QUIRÓS, Alajuela (CR); Establishment Labs S.A., Alajuela (CR)

(72) Inventors: Gerardo MORA, Alajuela (CR); Gerson RODRIGUEZ, Alajuela (CR); Raghu RAGHAVAN, Baltimore, MD (US); Martin BRADY, Baltimore, MD (US); Roberto DE MEZERVILLE, Alajuela (CR); Juan José CHACÓN QUIRÓS, Alajuela (CR)

(73) Assignee: Establishment Labs S.A., Alajuela (CR)

(21) Appl. No.: 17/282,495

(22) PCT Filed: May 30, 2019
(86) PCT No.: PCT/US2019/034667

§ 371 Date: Apr. 2, 2021

Related U.S. Application Data
(60) Provisional application No. 62/853,548, filed on May 28, 2019.
(60) Provisional application No. 62/740,861, filed on Oct. 3, 2018.

(51) Int. Cl. G16H 30/40; G06T 17/20; G09B 23/30

(52) U.S. Cl. CPC G16H 30/40; G06T 17/20; G06T 2210/41; G06T 2210/32; G09B 23/30

(57) ABSTRACT

Systems and methods are disclosed for processing images to determine a modified image The method may include receiving profile data associated with a subject, the profile data comprising three-dimensional image data corresponding to at least a portion of a torso of the subject. A three-dimensional model may be determined based on the profile data, and a breast volume model may be determined based on the three dimensional model.

### CROSS-REFERENCE TO RELATED APPLICATIONS

This application claims priority to U.S. Provisional Application No. 62/740,861, filed on Oct. 3, 2018, and U.S. Provisional Application No. 62/853,548, filed on May 28, 2019, the entireties of which are incorporated herein by reference.

### FIELD OF DISCLOSURE

The current disclosure relates to systems, methods, and computer-readable media useful for processing images, such as medical images, to determine one or more modified images.

### BACKGROUND

Implantable medical devices may be implanted into patients for a variety of reasons, including, for example, to improve the clinical condition of a patient, to replace natural patient tissue, or for aesthetic purposes. In many cases, implantable medical devices are implanted in patients having severe, complex, or chronic medical conditions. For example, breast implants may be used in reconstructive surgeries following mastectomies, e.g., after a cancer diagnosis, surgical removal of breast tissue, radiation therapy, and/or chemotherapy. Breast implants may also be used in surgery for aesthetic purposes, for example in augmentation or reduction mammoplasty (changing the size of the breasts).

In some such procedures, a plastic surgeon inserts a suitable implant at a desired region of the subject's body. In some cases, the subject may have to wait for the conclusion of the procedure to visualize the results of the procedure. Embodiments of the current disclosure may alleviate the problems discussed above and/or other problems in the art.

### SUMMARY

Embodiments of the present disclosure relate to systems, methods, and computer-readable media useful for medical procedures, including, e.g., cosmetic and reconstructive surgery related to the breast. Various aspects of the present disclosure may be useful for planning, simulating, and/or evaluating the outcome of cosmetic surgery, reconstructive surgery, and/or other medical procedures.

Systems, methods, and computer-readable media are disclosed that execute instructions for processing images to determine a modified image, comprising receiving profile data associated with a subject, the profile data comprising three-dimensional image data corresponding to at least a portion of a torso of the subject. A three-dimensional model may be determined based on the profile data, and a breast volume model may be determined based on the three dimensional model, the breast volume model comprising a plurality of tetrahedra. A reference tetrahedron may be determined for each tetrahedron of the plurality of tetrahedra of the breast volume model, each reference tetrahedron corresponding to a state of zero strain and zero fiducial total potential energy. An elastic potential energy of each tetrahedron of the plurality of tetrahedra may be determined of the breast volume model, and a total elastic potential energy of the breast volume model may be determined based on the elastic potential energy of each tetrahedron of the plurality of tetrahedra of the breast volume model. A resting equilibrium position of the breast volume model may be determined by minimizing the total elastic potential energy of the breast volume model, and a modified three-dimensional model of at least one breast of the subject may be determined based on the resting equilibrium position of the breast volume model.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate exemplary embodiments of the present disclosure. In these drawings, where appropriate, reference numerals illustrating similar elements are labeled similarly. For simplicity and clarity of illustration, the figures depict the general structure and/or manner of construction of the various embodiments. Descriptions and details of well-known features and techniques may be omitted to avoid obscuring other features. Elements in the figures are not necessarily drawn to scale. The dimensions of some features may be exaggerated relative to other features to improve understanding of the exemplary embodiments. For example, one of ordinary skill in the art appreciates that the cross-sectional views are not drawn to scale and should not be viewed as representing proportional relationships between different layers. Further, even if it may not be specifically mentioned in the text, aspects described with reference to one embodiment may also be applicable to, and may be used with, other embodiments.

FIG. 1 illustrates an exemplary imaging system and network environment according to techniques presented herein;

FIG. 2 is a flow chart that illustrates an exemplary method of scanning a subject using the imaging system of FIG. 1;

FIG. 3 is an exemplary aspect of a graphical user interface that may be generated using one or more systems of FIG. 1, and according to techniques presented herein;

FIG. 4 is an exemplary torso scan that may be displayed on a graphical user interface according to techniques presented herein;

FIG. 5 is an exemplary measurement data graphical user interface that may be displayed according to techniques presented herein:

FIG. 6 is an exemplary procedure simulation graphical user interface that may be displayed according to techniques presented herein;

FIG. 7 is an example graph of triangulation of the x,y plane according to techniques presented herein:

FIG. 8 is an example torso displaying tension lines in accordance with techniques presented herein:

FIGS. 9A-9C illustrates triangles that may be used in the construction of a geometry, according to techniques presented herein;

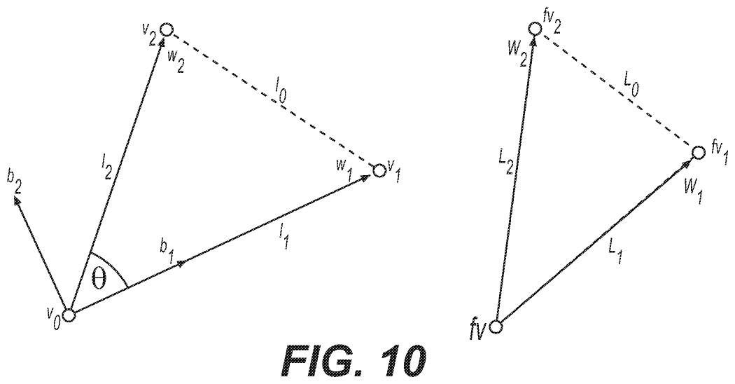

FIG. 10 displays example triangle geometries for simulating skin, according to techniques presented herein;

FIG. 11 displays the internal structure of the breast tissue, which may be referenced elsewhere herein;

FIG. 12 displays example tetrahedra representing an implant overlapping with tetrahedra representing breast tissue, according to techniques presented herein:

FIG. 13 displays example triangles with a property of mean bending, according to techniques presented herein;

FIG. 14 illustrates a non-conformal mapping of a change in structure due to tangential strain;

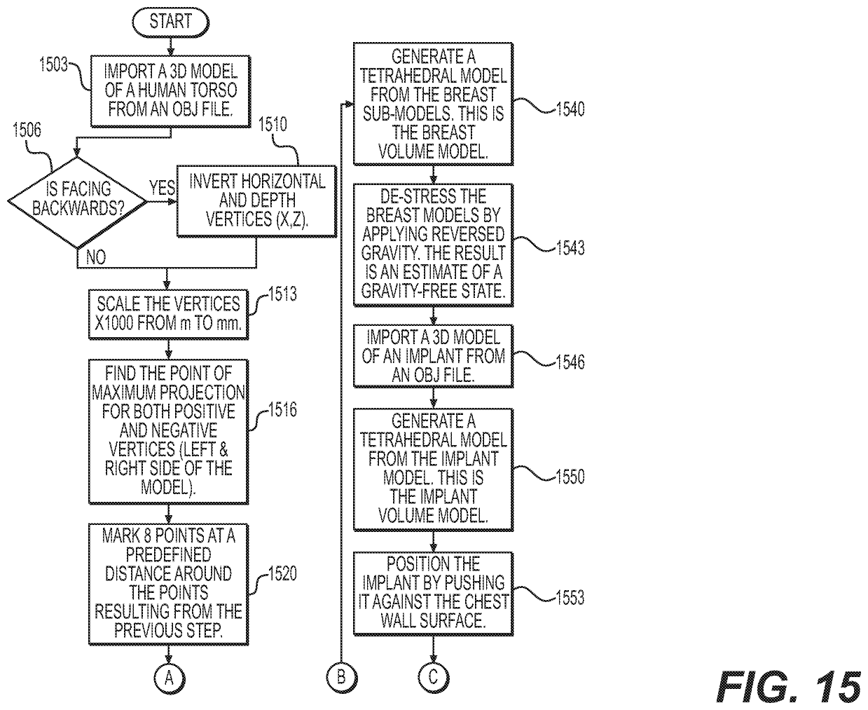

FIG. 15 is a flow chart illustrating an example determination of a three-dimensional breast model, according to techniques presented herein:

FIG. 16 is a flow chart illustrating an example determination of tangential relaxation, according to techniques presented herein;

FIG. 17 is a flow chart for determining a three-dimensional breast model, according to techniques presented herein; and

FIG. 18 is a simplified functional block diagram of a computer configured as a device for executing the methods of FIGS. 1-17, according to exemplary embodiments of the present disclosure.

### DETAILED DESCRIPTION

Aspects of the present disclosure are described in greater detail below. The terms and definitions as used and clarified herein are intended to represent the meaning within the present disclosure. The terms and definitions provided herein control, if in conflict with terms and/or definitions incorporated by reference.

In the discussion that follows, relative terms such as “about,” “substantially,” “approximately,” etc. are used to indicate a possible variation of 10% in a stated numeric value. It should be noted that the description set forth herein is merely illustrative in nature and is not intended to limit the embodiments of the subject matter, or the application and uses of such embodiments. Any implementation described herein as exemplary is not to be construed as preferred or advantageous over other implementations. Rather, the term “exemplary” is used in the sense of example or illustrative. The terms “comprise,” “include,” “have,” “with,” and any variations thereof are used synonymously to denote or describe nonexclusive inclusion. As such, a process, method, system, or device that uses such terms does not include only those steps, structure or elements but may include other steps, structures or elements not expressly listed or inherent to such process, method, system, or device. Further, terms such as “first,” “second,” and the like, if used herein, generally do not denote any order, quantity, or importance, but rather are used to distinguish one element from another.

In some embodiments, the current disclosure may include an imaging system that may be used, e.g., in cosmetic or reconstructive surgery (or another medical procedure). The imaging system may be used to visualize and/or simulate expected changes in the subject's appearance resulting from implantation, removal, or replacement of a breast implant. Further, for example, the imaging system may be used to design a custom breast implant based on at least one or more parameters of size, shape, surface texture, gel elasticity, gel rheology and/or shell elasticity of the implant. The imaging and simulation techniques herein may be used to select desired implant parameters and simulate the effects of the custom implant on a subject's body. Techniques discussed herein may further be used to simulate a medical procedure and/or anticipated result from a medical procedure, such as a mastopexy procedure, as will be discussed further herein.

According to some aspects of the present disclosure, for example, a selected manufacturer and model of breast implant, or a custom breast implant, may be represented as a tetrahedral model according to the techniques described below. The tetrahedral model may have model parameters (e.g., elastic moduli for each tetrahedron which may vary for different tetrahedra) based on the one or more of the parameters of size, shape, surface texture, gel elasticity, gel rheology and/or shell elasticity of the desired implant, so as to realize a simulation of the breast implant. This simulation then may be manipulated or tested in accordance with the imaging methods described herein. The simulation may include one or more variables or parameters, such as gravitational tissue relaxation factor, tissue elasticity, skin elasticity, chest wall asymmetry, surgical procedure, implant dimensions, and/or implant position.

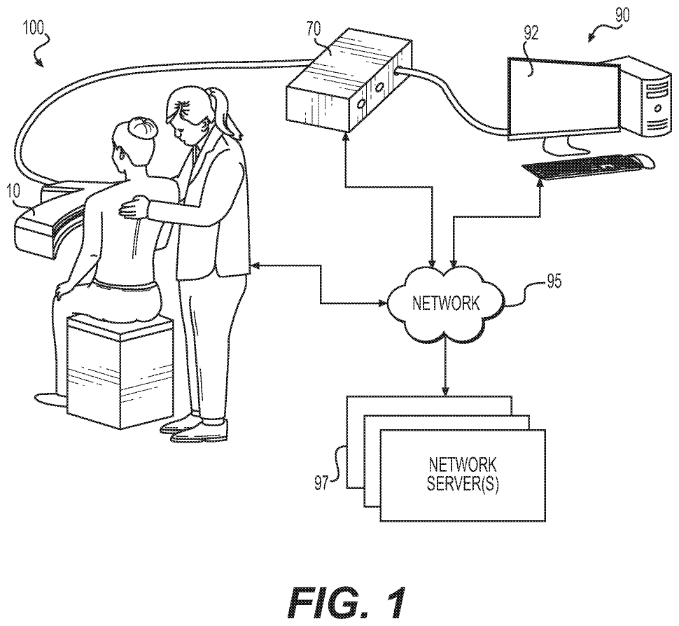

FIG. 1 illustrates an exemplary imaging system and network environment 100 of the current disclosure. The imaging system 100 may include an imaging scanner 10 (hereinafter scanner 10) configured to obtain one or more digital images of a subject (for e.g., a human patient considering various medical procedures) positioned in front of the scanner 10. The scanner 10 may be coupled to one or more controllers 70 configured to manipulate and control the operations of the scanner 10. The imaging system 100 may also include a computer system 90 and/or one or more network server(s) 97, operatively coupled to the controller 70 and the scanner 10. When discussing operations of the computer system 90 herein, it should be understood that one or more of these operations may be performed instead by the one or more network servers 97. The computer system 90 may be configured to direct the operations of the controller 70 and/or the scanner 10, either directly or over network 95, and receive image data from the scanner 10. The computer system 90 may be directly coupled to the controller 70 and/or scanner 10, or may be indirectly coupled via network 95. The computer system 90 may assist in creating a 3D digital image or model of the subject (akin to a digital body cast) using the data received from the scanner 10. In some embodiments, both the computer system 90 and the controller 70 may both be part of, or embedded in (e.g., physically one component), the scanner 10. In some such embodiments, the display device and a user input device (keyboard, mouse, etc.) may be coupled to the integrated scanner 10, for example, using cables. One or more network server(s) 97 may additionally communicate with the scanner 10, controller 70, and/or computer system 90 to control these devices, receive data from these devices, and/or assist one or more of these devices with processing, computation, storage, etc.

In the discussion that follows, the computer system 90 and the controller 70 are described as separate components. However, this is only exemplary. In some embodiments, both the computer system 90 and the controller 70 may be one component. For example, the features and functions of the controller 70 may be incorporated into the computer system 90, or the features and functions of the computer system 90 may be incorporated into the controller 70.

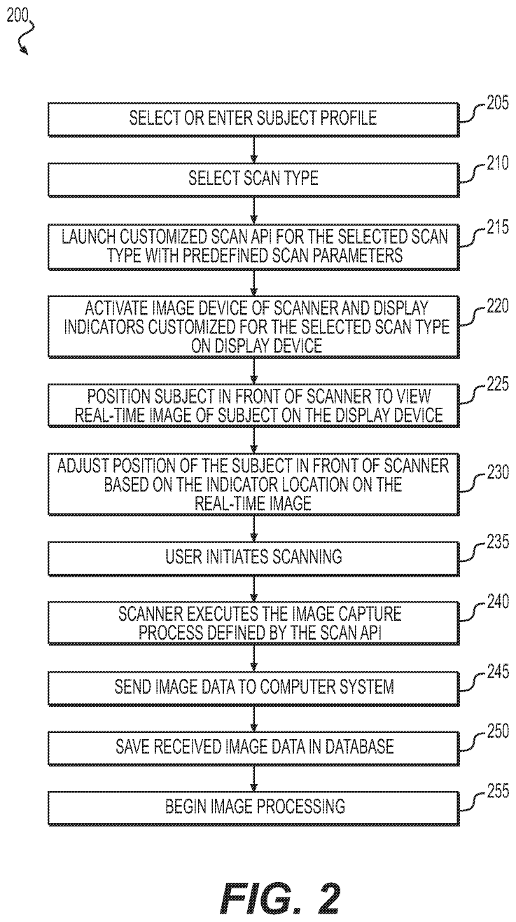

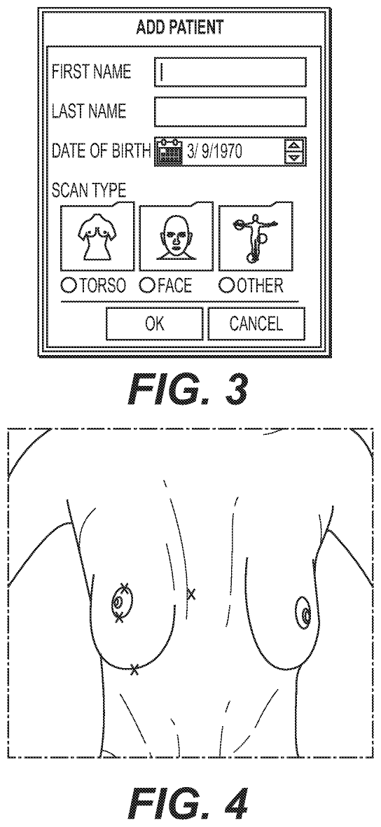

FIG. 2 is a flow chart that illustrates an exemplary method 200 of scanning a subject using a scan routine. Exemplary corresponding steps may be seen in International Publication No. WO 2017/175055, the entirety of which is incorporated by reference herein. The user may first select or create the subject's profile on the computer system 90 (step 205). In some embodiments, the names of existing subjects (e.g., clients or patients) may be presented on the display device 92, and the user may click on the subject's name to select the subject's profile. If there is no existing profile for the subject (e.g., no prior scans of the subject have been done), a new subject profile may be entered into the computer system 90 using an input device (e.g., keyboard, mouse, touchscreen, etc.). FIG. 3 illustrates an exemplary prompt generated by the computer system 90 for adding a new subject profile. After entering and saving the subject's profile (name, date of birth, etc.), the user may begin a scan.

In some embodiments, a pop-up window (or GUI) asking whether the user wants to begin a new scan or view an old scan (e.g., view the images associated with a previous scan) may be presented to the user. The user may begin a new scan by clicking an associated icon on the display device 92 (e.g., by clicking on icons labelled “begin new scan,” and/or “OK”). In some embodiments, the computer system 90 may prompt the user to confirm the prior selection. For example, a pop-up window asking “do you want to perform a scan?” may appear on the display device 92, and the user may click “yes” to continue with a new scan. The computer system 90 may then query the user to select the scan type (step 210). For example, the user may be presented with a pop-up window with a list of customized scan routines programmed into the computer system 90 (e.g., torso scan, facial scan, full body scan, etc.). Upon selection of the desired scan type, the computer system 90 may launch a scan API associated with the selected scan type that may have default (or preprogrammed) values for any parameters required by the scan API (step 215). These parameters may include the width, height, and depth of the imaging area, the lighting pattern (e.g., LED lighting pattern to achieve a desired cool/warm lighting pattern), intensity of lights, etc. For example, if the user selects “torso scan” as the desired scan type (i.e., in step 210), the scanner API for torso scan may be launched (in step 215) with default values for the parameters needed by the API. In some embodiments, the user may change the default values of the parameters in the API.

The launched scanner API may then activate the scanner 10 and display indicators (e.g., augmented reality target lines) associated with the selected scan type on the display device 92 of computer system 90 (step 220). The user then positions the subject in a scanning area in front of the scanner 10, and the computer system 90 may display a real-time image of the subject on the display device 92 (step 225). If the displayed indicators are not located at the appropriate locations of the subject's real-time image, the user may adjust the relative position of the subject with respect to the imaging device (step 230). For example, if the indicator for the sternal notch does not overlap with the image of the subject's sternal notch (on the display device 92), the imaging device and/or the subject may be repositioned until the indicator and the image overlaps. The indicator may also be a silhouette of the human body or portion thereof, for example a torso.

After the subject is properly positioned (in step 230), the user may initiate the scan routine in the computer system 90 (step 235). In some embodiments, the user may click on a “start scanning” button/icon of a graphical user interface (GUI) to initiate scanning. When the user initiates the scan routine (in step 235), the computer system 90 may execute instructions encoded in the scanner API launched in step 215. In response to these instructions, the scanner 10 may execute the image capture process defined by the scanner API (step 240). In some embodiments, the scanner API may send instructions to the controller 70 and/or the cameras (e.g., using serial communications) to start the selected scan type.

The scanner 10 may then send the acquired image data to the computer system 90 (step 245). In some embodiments, the computer system 90 may receive the image data from the imaging device in a real-time manner (i.e., as the scan is occurring). In some embodiments, the imaging device or the controller 70 may buffer (or store) the real-time image data and send the buffered data to the computer system 90 in a periodic manner or at the end of the scan. When the computer system 90 receives the image data from scanner 10 (e.g., .OBJ, .MTL, .PNG, .JPG files, explained below), the files may be saved in a database (locally or remotely located) and associated with the subject's profile (step 250). In some embodiments, after completing a scan, the computer system 90 may create a 3D image of the subject's scanned feature (e.g., torso) by reconstructing the image from the received image data, and display the reconstructed image on the display device 92 for the user to view. If the user is satisfied with the scan, the image processing process may start in computer system 90 (step 255).

Computer system 90 and/or network server(s) 97 may process the image data from the scanner 10 to create a 3D image (or digital 3D model) of the subject's scanned feature(s), and prepare the 3D image for simulations. The computer system 90 may retrieve and process image data saved in the database, or may process the image data as it is received from the scanner 10. In some embodiments, image processing may include converting separate image data files into a single file that contains all the data to produce the 3D image. For example, the scanner 10 may collect and save image data in different file formats. These file formats may include an OBJ file, a MTL file, and a PNG file (e.g., a files having extensions of .OBJ, .MTL, and .PNG, respectively), and/or other data in other file formats suitable for storing, transferring, and/or transmitting image data.

In the example wherein the 3D image is reconstructed from OBJ, MTL, and PNG files, the OBJ file may include mesh data for the 3D image. This file may include some or all the volumetric information for reconstructing the 3D image of the scanned feature. The PNG file may include information regarding texture and color, for example, of different regions of the scanned feature. And, the MTL file may include the configuration information (e.g., the relationship between the PNG and OBJ files). For example, for a torso scan, the PNG file may contain details related to color, texture, etc. of the skin in different regions of the subject's torso. In some embodiments, the MTL file may include the coordinates for proper alignment of the 3D mesh and colors/textures to produce a 3D image. The PNG file may include texture and color information for the scanned subject.

In some embodiments, image data obtained by the scanner 10 (e.g., OBJ, PNG, and MTL files) may be converted to a single file in an AX3 format (e.g., file having a file extension of .AX3) by computer system 90 (using software). In some embodiments, the AX3 file may convert and compress the data for storage as a hexadecimal Binary Large OBject (BLOB). The AX3 file may contain some or all the information necessary to generate a 3D image of the scanned feature. For example, the AX3 file may combine and store data from the OBJ, MTL, and PNG files, each of which includes a different type of image related information.

The computer system 90 may recreate and display a 3D image (or digital 3D model) of the scanned feature using the AX3 file (or other similar imaging file), and display the image on the display device 92. The computer system 90 may then prompt the user to identify or mark various anatomical features relevant to the area of the subject's body featured in the displayed 3D image. In some embodiments, the computer system 90 may display different views of the digital 3D model on the display device 92 in sequence, and prompt the user (e.g., using a GUI or one or more pop-up windows) to mark (or otherwise identify) relevant anatomical features on the displayed images.

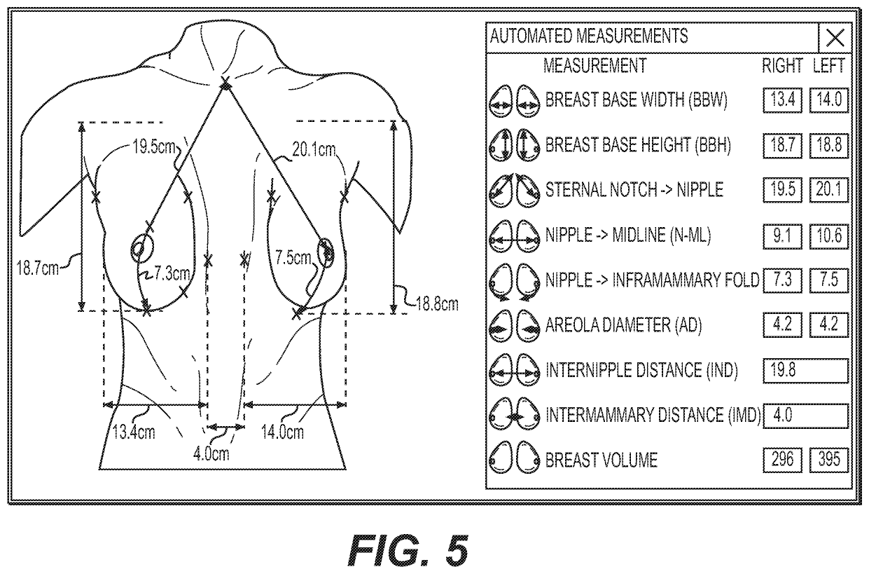

FIG. 4 illustrates an exemplary image from a torso scan of a subject indicating some of the anatomical features marked by the user on the image (marked “X” in FIG. 4). The computer system 90 may first display an image of the torso and prompt the user to mark (or otherwise identify) the location corresponding to a selected feature (e.g., right lateral) on the image.

In a similar manner, the computer system 90 may prompt the user to identify different anatomical features (e.g., all features that are relevant for the analysis) on the torso. These features may include some or all of right lateral, right bottom, right nipple, right areola radius, right sternum, sternal notch, left sternum, left bottom, left nipple, left areola, left lateral, etc. As each feature is marked by the user, the location of this feature on the displayed images may be indicated with a marker (and/or text labelling the identified feature, in some embodiments) identifying the location. After all the relevant anatomical features are identified or marked, the user-identified locations of all the marked features may be illustrated on an image, and the user may be prompted to approve, change, and/or save the identified locations (e.g., by pressing save, etc.).

In some embodiments, as successive images are displayed, the computer system 90 may calculate and display various parameters, based on the user's input, pictorially or as text on a displayed image (or elsewhere on the display device 92). For example, after the user identifies or marks the right sternum location, the computer system 90 may calculate the volume of the left breast based on the locations of the previously identified features and display this volume on the image (e.g., the next displayed image of the torso). Alternatively, or additionally, in some embodiments, the computer system 90 may identify or highlight an area encompassed by the locations of the previously identified features on the image.

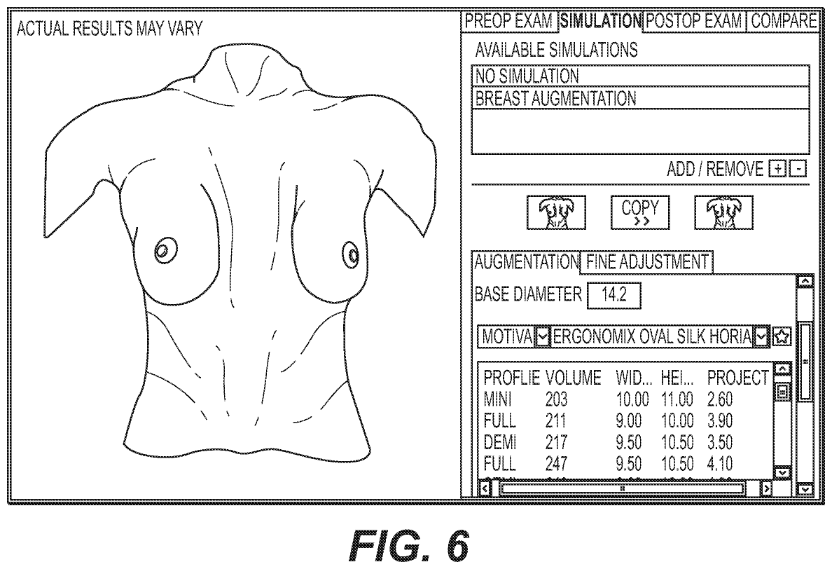

In some embodiments, as illustrated in FIG. 5, automatically computed parameters may be pictorially presented on the display device 92. In general, the results of the simulations may be presented in any form. In some embodiments, the simulation results may be presented on the display device 92 (e.g., textually, pictorially, etc.) of the displayed results may include the calculated tissue volume of the left breast and the right breast obtained from a simulation. In some embodiments, as illustrated in FIG. 5, a summary of the automated measurements, and the calculated values for the tissue volume of the left and right breasts may also be presented on the display device 92.

The subject's existing breast implant, and type of placement of the implant (sub-pectoral, sub-glandular, etc.), may be identified. In some embodiments, the computer system 90 may extract this information from the subject's profile, or acquire the information from the embedded implants using an RFID sensor of scanner 10. In some embodiments, the computer system 90 may prompt the user to enter this information (e.g., via pop-up windows, etc.). In some embodiments, the user may be prompted to select the type of implant from a database which includes information about different implants generally available, or from a list of implants presented on display device 92. Based on the information (dimensions, etc.) of the existing implant, the computer system 90 computes or defines a virtual implant in the reconstructed 3D model of the subject's torso, and presents the data. For example, based on the volumetric and placement information of the subject's existing implant, the computer system 90 may attribute a volume in the reconstructed 3D model as being occupied by the implant, and present this data to the user. Although the data may be presented in any manner, in some embodiments, the computed existing implant volume and location may be identified in a digital 3D model of the subject's torso displayed on the display device 92, as will be discussed further below.

If the user is satisfied with the represented virtual implant, the user may activate an implant removal simulation function in the computer system 90. In some embodiments, the user may click on a button or an icon presented on the display device 92 to activate or launch an algorithm (or subroutine) associated with the implant removal function. This algorithm may execute a virtual tissue removal function, and perform simulations that reduce the overall volume of the breast by an amount equal to the existing implant volume, to simulate the removal of the implant from the breast. The results may then be presented to the user, for example, on the display device 92.

Following the simulations discussed above, which may involve simulating the removal of an existing implant, as will be discussed further herein, the user may perform other simulations, such as, for example, simulation of breast augmentation surgery. Breast augmentation is a surgical procedure where a breast implant is placed under the subject's chest muscles or breast tissue to increase the size, shape, and/or fullness of the breast. Knowing the type of implant that a subject is considering, breast augmentation surgery simulations may create a virtual 3D model of the implant, reconstruct the 3D model of the subject's torso with the implant embedded therein, and present the results to the user. The presented results may include a digital 3D model of the subject's torso (e.g., a modification of the original 3D image obtained using the scanner 10 or obtained as a result of a previous simulation) with the selected implant embedded therein. Based on these results, the subject can decide to go ahead with the surgery or select another implant or placement/orientation information for simulations.

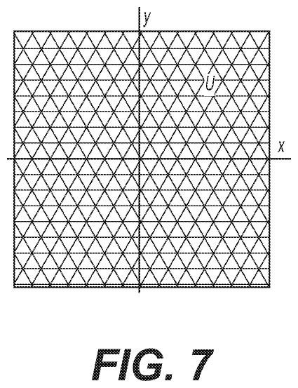

In a breast augmentation surgery simulation, the user (or subject) may first select an implant for the simulations and provide details of the implantation procedure (e.g., sub-pectoral implantation, sub-glandular implantation, etc.) to the computer system 90. As explained previously, the details (dimensions, model, etc.) of the implant may be provided by the user to the computer system 90, or the implant may be selected from a database that includes details of different types of implants. FIG. 6 is an illustration of a database that includes an exemplary list of available implants of varying sizes (e.g., mini, full, demi, corse, etc.). In some embodiments, the computer system 90 may generate a list of implants that takes into account particulars of the subject (e.g., the dimensions of the torso, volume and/or type of existing breast tissue, etc.). For example, the computer system 90 may exclude from the presented list, implants that fall outside a range of implant sizes determined to be suitable for a given subject (e.g., based on defined criteria). The user may select a desired implant from the presented list of available implants (e.g., from the list of FIG. 6). As will be discussed further below, using details of the implant and the implantation procedure, the simulation algorithm may compute a virtual implant and reconstruct the digital 3D model of the torso with the implant embedded therein. The results of simulations may be presented on the display device 92. The user may review and validate the results, or modify the parameters and initiate re-computation with the modified parameters.

In general, the effect of any type of breast tissue/implant/prosthesis removal or addition may be simulated using imaging system 100. In some embodiments, simulations may be performed to visualize and study the effect of breast reconstruction following a mastectomy or partial mastectomy (e.g., due to breast cancer). In addition to breast implants, in some embodiments, the imaging system 100 may be configured to simulate tissue expanders used to prepare chest tissue to receive an implant. For example, the imaging system 100 may perform volumetric calculations that take into account a subject's existing breast tissue, and then allow the user to simulate one or more tissue expanders and/or one or more breast implants following the tissue expanders(s) to be used during breast reconstruction surgery.

Simulating the Results of Surgical Procedures

Techniques for simulating possible surgical procedures for the breast will now be discussed. There is a need for computational models of an elastic material (such as breast and breast-cum-implant) that can respond fast enough for interactive applications, while retaining a clear relation with physical theory. According to some aspects herein, the response time may be computed on the order of milliseconds, e.g., providing an interactive experience for the user (e.g., subject) as compared to autonomous simulations run with predefined input for examining the results afterward. Interactive physical modeling may employ algorithms that do not rely on a network of springs (which may poorly represent such factors as shear resistance, or incompressibility of area), yet speed may still be emphasized over decimal places of accuracy. This application describes techniques for simulating the mechanics related to breast tissue and breast prosthesis procedures based directly on their simplicial subdivision. These techniques model the mechanics of individual (patient-specific) tissue sufficiently accurately to be useful in predicting the outcome of surgeries.

The methods herein may employ data specific to a subject, e.g., which may provide for a more realistic simulation. For example, one or more characteristics associated with the subject may be retrieved from a profile, e.g., a subject-specific profile. Exemplary profile data may include, but are not limited to, a gravitational tissue relaxation factor, tissue elasticity, skin elasticity, chest wall asymmetry, or a combination thereof. Further, the profile data may include contact information for the subject's physician or other medical professional and/or the date(s) and type(s) of medical procedures previously performed. According to some aspects of the present disclosure, the profile data may include whether the subject has an existing implant and information about the implant(s) (such as, e.g., manufacturer, model, type, size, age, integrity, volume, surface texture, a unique device identifier (e.g., RF transponder), and/or type of filling material (e.g., silicone gel), among other implant parameters). The profile data optionally may be stored in a database, e.g., and retrieved before, after, and/or during processing of images as part of a simulation. For example, the profile data may be saved on a local network and/or in a cloud-based network.

There may be at least four components to the model in techniques related to breast tissue and breast prosthesis implantation procedures discussed herein:
1. Rigid skeletal bases such as the chest wall (e.g., wherein parameters may be marked as fixed).
2. The surface skin, treated as a flexible and extensible two-dimensional surface.
3. Bulk tissue, wherein the fine structure may be ignored (blood vessels, ligaments, etc.). Each tetrahedron in the subdivision may be treated as a uniform homogeneous (though not necessarily isotropic) material.
4. Introduction of foreign materials such as filler, implants, and so on, which may be treated separately for each class of application.
   According to some aspects of the techniques herein, the geometry assigned to these model components is first specified, then the material properties, and finally the mechanics.

For example, the geometry of model components, material properties, and mechanics may be used to determine a three-dimensional model of at least a portion of a subject's body, e.g., based on profile data associated with the subject (which may include three-dimensional image data obtained through scans as described above). This three-dimensional model may be used to determine a breast volume model, wherein parameters of the breast volume model may be modified, e.g., mathematically manipulated according to the methods herein, to determine a modified three-dimensional model. As further described below, the modified three-dimensional model may include incorporating a breast implant model (e.g., reflecting parameters of a contemplated size, shape, volume, style of breast implant) into the breast volume model. Thus, for example, the models encompassed herein may allow a subject to simulate the subject's appearance following an implantation procedure, including, e.g., natural movement of tissue, stretching of skin, effects of gravity on tissue, etc.

Geometry—Coordinates

Coordinates for physical space may be taken as orthogonal (x, y, z), with x lateral across the patient's chest, y from abdomen to chin, and z forward from the chest: of two points with the same (x, y), the one with the higher z will be called z-above the other. This frame, together with the standard inner product, may be referred to as E³. The standard inner product of two vectors v and w will be denoted either by v·w or by g(v,w), where multiple inner product functions are in question.

Geometry—Position in Space

A position ƒ of the model may be fully specified by a list of the (x, y, z) coordinates for each vertex: the positions of all edges, triangles and tetrahedra may follow. A change of ƒ to another position ƒ′, representing a motion or deformation. Lengths, angles, areas, etc., may be changed. This is distinct from the unchanging ‘reference geometry’ described below, which may be fixed throughout the calculation.

In general one may require that ƒ be an ‘embedding’ (e.g., no two ‘simplices’-points, edges, triangles, tetrahedra—have points in common that do not follow from sharing them in the reference model), and take steps to check this as the code evolves ƒ. It may be expected to be true, and remain so, without slowing the code to check.

Geometry—Rigid Boundaries

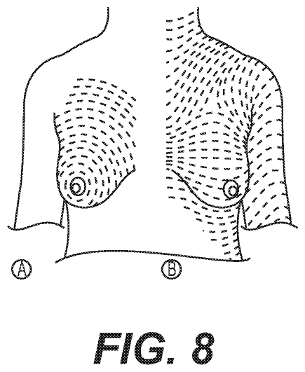

The model may accept an estimate of rigid surface boundaries such as the chest. This might, for instance, be extracted from a scan, or may be taken from a predetermined set of shapes of the chest that may be matched to the scan, or judged by eye from a technician to fit the patient. Geometries in techniques presented herein may be triangles or tetrahedra, e.g., so this surface may be triangulated. For example, a plurality of tetrahedra may be used to describe a breast volume model, and a plurality of triangles may be used to describe a breast surface model, associated with the breast volume model (e.g., a breast surface model comprising a plurality of triangles common with corresponding surface triangle faces of a plurality of tetrahedra of the breast volume model). Small regions may be described as a “height map” zₛ(x, y), and it may be adequate to take regular triangles (side length one or a few mm) on the relevant part U of the xy plane (see FIG. 7), with vertices (xᵢⱼ, yᵢⱼ), and use the corresponding triangles with the corresponding vertices (xᵢⱼ, yᵢⱼ, zₛ(xᵢⱼ, yᵢⱼ)). As the region U need not be rectangular, it may be more convenient to refer to vertices by a single index k than by array indices i and j.

The topology and geometry data for this component may include, besides whatever lists of edges, etc., are useful for faster computing:
The set of vertices vₖ, indexed by a list index k, with their double precision coordinates (x, y, z); and
the set of triangles Tₗ^{c}, with the indices of their corners kₒ(l), k₁(l), k₂(l): not directly (x, y, z) data.
   Note that this list may be reduced after the skin geometry is modeled.

Geometry—Skin

The skin may be represented in the model (e.g., in a breast surface model) as sets of triangles and vertices, but acquiring these models may be difficult. If gravity has a large effect on the shape, gravity may be normalized. The topology data for this component, the model triangular mesh Mₛₖᵢₙ of the skin, may include the sets of:

1. Vertices vᵢ^{skin}, indexed by a list index i.

2. Triangles Tᵢ^{s}, with the indices k=e^{s}ₒ(l), e^{s}₁(l), e^{s}₂(l) in #3 of their edges. 3. Edges Eₖ^{s}, with the indices of their ends k₀^{skin}(l), k₁^{skin}(l).

This mesh may end at a curve somewhat z-above the rigid model surface. Its edge Eₛₖᵢₙ=∂Mₛₖᵢₙ (union of the triangle edges unmatched by neighboring triangles) may be extended to reach the mesh Mₛₜ of that surface. One method is to find the nearest rigid surface vertex to each vertex of Eₛₖᵢₙ, identify the shortest loop \tilde{E} of surface triangle edges that passes through all these points, and connect Eₛₖᵢₙ and \tilde{E} with a ribbon of triangles. The triangles of the rigid surface that lie outside \tilde{E} may then be discarded. Topologically, with the first approach above, this is the structure of the (pruned) set of triangles in (x, y) space. Next, the reference state of a triangle T₁^{s} derived from the reference state of its edges will be discussed.

The parameter l(eₖ^{s}), or lₖ^{s}: for short, may be defined as the distances between the ends of edge eₖ^{s} if and when the skin around them is relaxed. A small separated piece of skin, as in a skin flap removed for graft elsewhere, approximates this relaxation.

As long as it is on the body, the skin is not relaxed. Indeed, the shrinkage when a graft is removed from the donor site typically includes removal of a larger area than the target where it will be sutured in, in view of differences in tension.

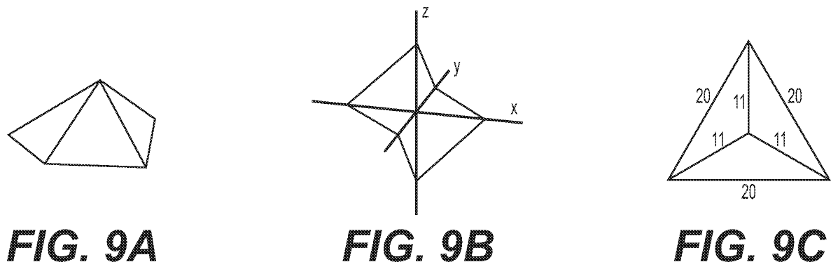

Moreover, there are cases in which the tension is not isotropic. Herein the term “Langer lines” or skin tension lines is used to refer to the lines of maximum extension of the skin, as suggested by Langer's original experiments. FIG. 8 illustrates lines a and b of maximum resting skin tension in the breast (also referred to as dynamic lines of Kraissl). The direction field may be denoted by a unit tangent vector l(p) at each point p on the averaged surface (defined up to sign). An estimate of the unit normal n(p) gives the unit tangent vector l^{⊥}(p) as l(p)×n(p). It may be desired to have the amount of stretching a(p) along the major axis of Langer's elliptical hole at p, and similarly for b(p) along the minor axis. These can be measured by appropriate devices, or estimated from typical Langer lines and stretch values for the patient's demographic. (e.g., age reduces stretch values: pregnancy or an implant temporarily increases them, but long-term stretch leaves values lower than before once the cause of the stretching is removed.)

If edge eₖ^{s}, from vᵢ^{skin} to v_{f}^{skin} has measured end positions pᵢ and pⱼ; there is (approximately) a tangent vector e̅i̅j̅=pⱼ−pᵢ, which may be decomposed as described by equation (2):

e̅i̅j̅=(e̅i̅j̅×1)1+(e̅i̅j̅×1^{⊥})1^{⊥} (2)

This is stretched from orthogonal components of length (e̅i̅j̅×1)/a and (e̅i̅j̅×1)^{⊥}/b, so the reference length may be set according to equation (3):

$${l}_{k}^{s} = l ( {E}_{k}^{s} ) = \sqrt{{( \frac{\overline{{e}_{ij}} \cdot 1}{a} )}^{2} + {( \frac{\overline{{e}_{ij}} \cdot {1}^{⊥}}{b} )}^{2}} . \qquad ( 3 )$$

These lengths determine the reference shape of the triangles. Skin has been under tension rather than in a relaxed state since before birth, through all the changes of shape in development, and may grow with such reference lengths. Skin is understood to adapt to the growing flesh it contains.

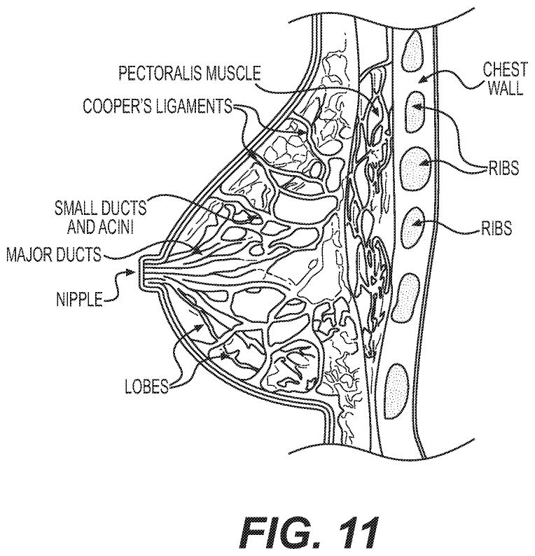

The corresponding triangle in [symbol] ³, by some position ƒ, is shown on the right of FIG. 10. As ƒ changes the computation, typically in steps from an initial position obtained by guess or measurement to an equilibrium one, this triangle denotes the reference triangle's position in [symbol] ³, which without ƒ, may not have one. Denoting the reference triangle and its sides by Tₗᵢ, l₀, l₁, and l₂ for short, and the vertices, v₀, v₁ and v₂, the geometry data for the mesh Mₛₖᵢₙ may include:
The double precision coordinates (xⱼ, yⱼ, zⱼ) skin of the vertices vᵢ^{skin} of the embedded, not the reference triangle.
The reference lengths l(Eₖ^{s}) of the mesh edges, lₖ^{s} for short.
The areas a₁=area(Tₗ^{s}) of the triangles, as implied by the reference lengths of their sides.

Unlike the (x, y, z) space in which the position data is recorded, in some cases the bᵢ b₂ plane in FIG. 10 does not have an origin or axes, though it may have a measure of lengths and angles. Choosing v₀ as the origin, the side vectors w₁ and w₂ may be as shown. Thus, according to equations (4)-(6):

$$\begin{matrix} & \\ {w}_{1} \times {w}_{1} = {l}_{1}^{2} & ( 4 ) \\ {w}_{2} \times {w}_{2} = {l}_{2}^{2} & ( 5 ) \\ {l}_{1} {l}_{2} cos θ = {w}_{1} \times {w}_{2} = \frac{{l}_{2}^{1} + {l}_{2}^{2} - {l}_{0}^{2}}{2} & ( 6 )\end{matrix}$$

by the cosine formula, with w₁ and w₂ as a basis W, the standard inner product on [symbol] ² has the matrix

$${G}_{w} = {[ \begin{matrix}{l}_{1}^{2} & \frac{{l}_{1}^{2} + {l}_{2}^{2} - {l}_{0}^{2}}{2} \\ \frac{{l}_{1}^{2} + {l}_{2}^{2} - {l}_{0}^{2}}{2} & {l}_{2}^{2}\end{matrix} ]}_{w} = {[ \begin{matrix}{g}_{11} & {g}_{12} \\ {g}_{12} & {g}_{22}\end{matrix} ]}_{w} \qquad ( 7 )$$

for short. To measure how a position ƒ distorts general vectors, it may be convenient beforehand to define also the orthonormal basis according to equation (8):

$$\begin{matrix}{b}_{1} = \frac{{w}_{1}}{‖ {w}_{1} ‖} & {b}_{2} = {b}_{1}^{⊥}\end{matrix} \qquad ( 8 )$$

where b₂ lies on the same side of b₁ as w₂. The computation of the reference areas a₁ can be done for each triangle in this basis. In this basis B, the same inner product has the matrix

$${G}_{B} = {[ \begin{matrix}1 & 0 \\ 0 & 1\end{matrix} ]}_{B} = {[ \begin{matrix}{δ}_{11} & {δ}_{12} \\ {δ}_{21} & {δ}_{22}\end{matrix} ]}_{B} \qquad ( 9 )$$

And w1, w2 may have the component forms

$${[ \begin{matrix}{w}_{1}^{1} \\ {w}_{1}^{2}\end{matrix} ]}_{B} = {[ \begin{matrix}{l}_{1} \\ 0\end{matrix} ]}_{B} , {[ \begin{matrix}{w}_{2}^{1} \\ {w}_{2}^{2}\end{matrix} ]}_{B} = {[ \begin{matrix}\frac{{l}_{1}^{2} + {l}_{2}^{2} - {l}_{0}^{2}}{2 {l}_{1}} \\ \sqrt{{l}_{2}^{2} - {( \frac{{l}_{1}^{2} + {l}_{2}^{2} - {l}_{0}^{2}}{2 {l}_{1}} )}^{2}}\end{matrix} ]}_{B} \qquad ( 10 )$$

The change-of-basis matrices from W to B and vice versa may be

$$\begin{matrix}{I}_{w}^{B} = {[ \begin{matrix}{w}_{1}^{1} & {w}_{2}^{1} \\ {w}_{1}^{2} & {w}_{2}^{2}\end{matrix} ]}_{w}^{B} , & ( 11 ) \\ {I}_{B}^{w} = {[ \begin{matrix}{\tilde{w}}_{1}^{1} & {\tilde{w}}_{2}^{1} \\ {\tilde{w}}_{1}^{2} & {\tilde{w}}_{2}^{2}\end{matrix} ]}_{B}^{w} = {( {I}_{w}^{B} )}^{- 1} , & ( 12 )\end{matrix}$$

respectively, where

I_{W}^{B}I_{W}^{B}=I_{B}, (13)

the identity matrix on vectors referred to [symbol] . Then

[symbol]

=( [symbol] )^{T} [symbol] (14)

[symbol]

=( [symbol] )^{T} [symbol] (15)

All the above may be setup calculations for the reference geometry. The geometry of ƒ may change as it iterates. It maps the ‘abstract’ reference vₒ, v₁, and v₂ to the vertices ƒvₒ, ƒv₁ and ƒv₂ in [symbol] ³ with the appropriate (x, y, z) coordinates (which v₀, v₁ and v₂ lack, not being in [symbol] ³).

There may be corresponding explicit vectors, shown in equations (16) and (17) as follows:

W₁=ƒ(v₁)−ƒ(v₀) (16)

W₂=ƒ(v₂)−ƒ(v₀) (17)

With the (x, y, z) basis [symbol] for vectors in [symbol] ³, on this triangle ƒ may have the matrices shown in equations (18) and (19)

$$\begin{matrix}{F}_{w}^{ℛ} = {[ {W}_{1} {W}_{2} ]}_{w}^{R} = {[ \begin{matrix}{W}_{1}^{x} & {W}_{2}^{x} \\ {W}_{1}^{y} & {W}_{2}^{y} \\ {W}_{1}^{z} & {W}_{2}^{z} ]\end{matrix} ]}_{w}^{ℛ} & ( 18 ) \\ {F}_{B}^{R} = {F}_{w}^{R} {I}_{B}^{w} & ( 19 )\end{matrix}$$

according to whether reference vectors are referred to [symbol] or [symbol] . The matrix of equation (20)

$${G}_{fw} = [ \begin{matrix}{W}_{1} \cdot {W}_{1} & {W}_{1} \cdot {W}_{2} \\ {W}_{1} \cdot {W}_{2} & {W}_{2} \cdot {W}_{2}\end{matrix} ] \qquad ( 20 )$$

expresses the standard inner product [symbol] ³ in the plane the embedded triangle occupies, in the basis {W₁, W₂}. However in computing energies, it may be helpful to describe the strain from the reference triangle in FIG. 10. Thus, to compare it with G_{b} in the shared basis [symbol] ={b₁, b₂}, it may be pulled back by the same transformations (derived from the reference lengths), as in equation (14) to produce equation (21):

[symbol]

=( [symbol] )^{T} [symbol] (21)

Comparing equation (5) and equation (20) shows how the lengths change. The matrix

Tₛₜᵣₐᵢₙ= [symbol] − [symbol] = [symbol] −I (22)

may express the deformation in a manner independent of the shape of the particular triangle. When ƒ changes (e.g., the model ‘moves’), only equations (20)-(22) represent new computations to update Tₛₜᵣₐᵢₙ. The matrices (I_{B}^{W})^{T} and (I_{B}^{W})^{T} may be fixed by the reference lengths.

Before describing approaches to bending and curvature energies, strain energies in three dimensions is discussed below.

Resting Geometries

The specifics of above techniques performing tissue simulations will now be discussed in greater detail. Gravity has a large effect on breast shape, which may need to be normalized. A microgravity environment is normally unavailable and may generate its own distortions. To get more information, it may be beneficial to scan the subject at least twice, for example, once with the body supine (lying face up) and the breast(s) unsupported, and a second time with the body prone (lying face down), with the breast(s) in a gap between support for the clavicle and abdominal regions. Additionally or alternatively, the subject may be scanned in an erect position (standing up). Such positions during a scan may be referred to herein as fiducial positions. A sitting scan may be coordinated with the prone, supine, and/or erect scans and corresponding points may be identified. This may provide a bulk estimate of ‘sag’, and hence elastic response, to improve the predictions of the final shape. This may be a supplement rather than a replacement.

In at least one embodiment, laser scans may generate surfaces zₛᵤₚᵢₙₑ(x, y) and zₚᵣₒₙₑ(x, y). More specifically, the scan software may generate at least a point cloud, and may then generate a triangulated mesh—that is, in this context, a piecewise linear (on triangular pieces) function z(x, y).

This approach may use equation (23)

$${z}_{skin} ( x , y ) = \frac{{z}_{supine} ( x , y ) + {z}_{prone} ( x , y )}{2} \qquad ( 23 )$$

as giving a less-affected shape, and triangulate it, for example, by the methods used in the rigid boundaries section above, or by the triangles given by the software for zₚᵣₒₙₑ(x, y) considered as a function, adjusting z on the vertices by the equation above, and the linear interpolation built into zₛᵤₚᵢₙₑ(x, y).

Because points move in x and y as well as vertically, this approach approximates the gravity-free shape (which may be better than taking the prone, supine, or sitting shape alone).

In another enablement, when prone and supine scan data are difficult to obtain, the resting gravity-free shape may be obtained by applying gravity in reverse as an upward force. The accuracy of this shape will depend on obtaining reasonable values for the effective elasticity of the tissue.

In some cases, scans may involve techniques similar to those used in integrating multiple photographic views and recreating a texture map of an object surface. For example, each point (x, y, z)_{scan} found may have other data recorded with it, such as color and/or texture information. This information may make it possible to identify for a point (xᵢ, yᵢ, zᵢ)ₛᵤₚᵢₙₑ the nearest point (\tilde{x}ᵢ, \tilde{y}ᵢ, \tilde{z}ᵢ)ₚᵣₒₙₑ with the same properties, and hence the same material point i on the skin, after deformation. A surface-to-surface fit, minimizing overall mismatch, may be desired in some cases, e.g., as opposed to a point-by-point match. The functions may then be described according to equation (24) denoted:

$${( {x}_{i} , {y}_{i} , {z}_{i} )}_{skin} = \frac{{( {x}_{i} , {y}_{i} , {z}_{i} )}_{supine} + {( {\tilde{x}}_{i} , {\tilde{y}}_{i} , {\tilde{z}}_{i} )}_{prone}}{2} \qquad ( 24 )$$

and used to triangulate this point cloud. The topology data for this component, the model triangular mesh Mₛₖᵢₙ of the breast skin, may include the sets of:
1. Vertices vᵢ^{skin}, indexed by a list index i.
2. Triangles Tₗ^{s}, with the indices of k=e₀^{s}(l), e₁^{s}(l), e₂^{s}(l) in #3 of their edges.
3. Edges Eₖ^{s}, with the indices of their ends k₀^{skin}(l), k₁^{skin}(l).

This mesh may end at a curve somewhat z-above the rigid chest model surface. Its edge Eₛₖᵢₙ=∂Mₛₖᵢₙ (union of the triangle edges unmatched by neighboring triangles) may thus be extended to reach the mesh M_{chest} of the chest. One method is to find the nearest chest vertex to each vertex of Eₛₖᵢₙ, identify the shortest loop \tilde{E} of chest triangle edges that passes through all these points, and connect Eₛₖᵢₙ and \tilde{E} with a ribbon of triangles.

The triangles of the chest that lie outside \tilde{E} may be discarded. Topologically, with the first approach above this corresponds to the structure of the (pruned) set of triangles in (x, y) space (FIG. 7).

Tetrahedral Geometry

In three dimensions, where tetrahedra replace the triangles of two dimensional spaces, analogous formulas may be straightforwardly written down by those skilled in the art. We thus have a corresponding strain tensor in three dimensions, following formulas fully analogous to those in Geometry-Skin above, but extending to 3-dimensional vectors and 3×3 matrices.

Implant Membrane

Breast implants come in varied shapes and internal structures. One possible technique for simulating the effects of various implants on breast tissue according to the methods herein is to ignore variations in internal compositions of the implants but not the shapes, e.g., the shapes having an impact on the outcome. This framework may take into account the mechanics and shear forces of the breast tissue material and/or the implant content.

As discussed above, according to some aspects of the present disclosure, the user may select an implant model, e.g., a breast implant model, from a list. Each model may have a geometric specification in reference pre-implantation coordinates (u, v, w) supplied, e.g., by the manufacturer, in a format such as IGES or STEP. Alternatively, the implant dimensions may be measured in-house, and a triangulated mesh may be generated. To transform (u, v, w) vertex coordinates into body coordinates (x, y, z), an intended position may be provided, given by, for example, a 4×4 matrix A specifying the rotation and translation by which it arrives at that location and attitude: (x, y, z, 1)=A(u, v, w, 1).

In use, this may be generated by a three-dimensional user interface, by which a surgeon can visually plan where to put the implant, relative to a three-dimensional rendering of the current skin geometry (obtained as in “Details of Skin Simulation” and “Skin Simulation” sections above). Again, this may be used when the flesh has shear forces. For implant removal/replacement, a scan of the implant in situ may be used to define even the volume of the implant (which may have changed, even if the catalogue number is known). One approach is an ultrasound scan, producing a triangulated mesh, similar to the skin. It may also be determined whether an implant changes shape significantly between the prone and supine positions of the subject's body.

The geometry data for this mesh Mᵢₘₚₗₐₙₜ may include
the set of vertices vⱼ^{brane}, indexed by a list index j, with their double precision coordinates (x, y, z); and/or
the set of triangles Tₗ^{m}, with the indices of their corners j₀(l), j₁(l), j₂(l).

The analysis of triangle deformation geometry above may apply equally here. Thus, for example, the methods of processing images herein may include receiving a breast implant model; determining a total elastic potential energy of the breast implant model; and determining a resting equilibrium position of the breast implant model by minimizing a total energy of the breast implant model using the total elastic potential energy of the breast implant model. The breast implant model may be based on one or more implant parameters. Exemplary implant parameters include, but are not limited to, implant size, implant shape, implant surface texture, elasticity of a gel filling material, rheology of a gel filling material, elasticity of a shell of the implant, and combination thereof.

Breast Volume

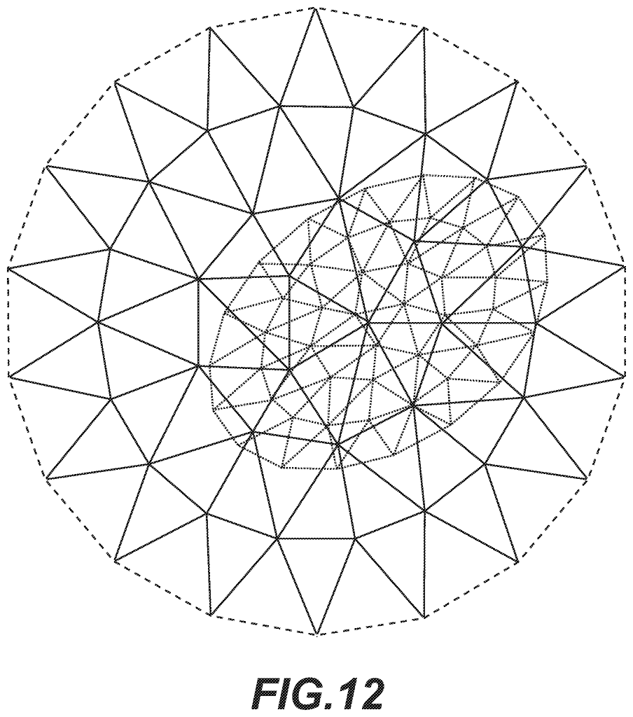

Breast volume refers to the space between the chest, skin, and surface of the breast implant (e.g., shell or membrane of a breast implant). For techniques discussed herein, the internal tissue structure shown in FIG. 11 may be ignored, including the Cooper's ligaments, blood vessels, etc. Instead, the closed-surface triangulated mesh may be taken from the union of M_{chest}, Mₛₖᵢₙ, and Mᵢₘₚₗₐₙₜ, points being scattered evenly a few millimeters apart, or some other predetermined density, and fill with tetrahedra.

The topology data for this mesh Mₜᵢₛₛᵤₑ may include lists of;
vertices vⱼ^{tissue}, indexed by a list index j;
tetrahedra Tₗ^{t}, with the indices of their corners j₀(n), j₁(l), j₂(l), j₃(l);
the triangles (tetrahedron faces) shared with the chest;
pointers to the triangles shared with the skin;
pointers to the triangles shared with the membrane; and/or
the reference volumes Vₗ^{t} of the tetrahedra Tₗ^{t}.

The volume of each Tₗ^{t} may be taken as ⅙ of the determinant of three edge vectors from a common corner, in terms of the coordinates in which the tetrahedralization was performed. The lengths of the edges need not be stored (except perhaps the edges shared with the skin and membrane triangles). The total volume of the breast (V_{B}) may be determined.

The compressibility of the breast tissue may be treated by examining the volume changes of the tetrahedra. However, it may be more customary to use the two independent invariants (whose coefficients are thus combinations of the isotropic bulk and shear moduli), namely Tr (Tₛₜᵣₐᵢₙ)² and Tr (T²ₛₜᵣₐᵢₙ). These may be used instead of a volumetric expression, which may be augmented with, for example Tr [Tₛₜᵣₐᵢₙ−I Tr (Tₛₜᵣₐᵢₙ)/3]². Nevertheless, there may be a distinction between these two expressions in the discrete case which disappears in the continuum limit. However, the direct expression of volume changes may serve another purpose, namely to inhibit inversion of tetrahedra. These considerations may apply to the implant volume as well. To assume invariance under rotation may take the skin's material properties (though not the strain) as isotropic. However, the natural orientation of collagen fibers in the dermis may make the elastic coefficients directional. Isotropy may be provisionally assumed, pending measurements of this directionality.

Implant Volume

The implant volume refers to the space enclosed within the implant shell or membrane Mᵢₘₚₗₐₙₜ. In at least one embodiment, the internal structure of the implant (e.g., specifics of a filler material or materials) may be ignored. Points may be scattered evenly within it, a few millimeters apart (or another predetermined distance/density) and filled with tetrahedra.

The geometry data for this mesh M_{stuffing} may include
the set of vertices vⱼ^{stuffing}, indexed by a list index j, with their double precision coordinates (x, y, z);
the set of tetrahedra Tₗ^{s}, with the indices of their corners j₀(l), j₁(l), j₂(l), j₃(l);
the sublist of triangles (tetrahedron faces) shared with the implant membrane, with pointers to the membrane triangles; and/or
the reference volumes Vₗ^{s} of the tetrahedra Tₗ^{s}.

Let the total volume of the implant be denoted V_{I}.

Implant Insertion

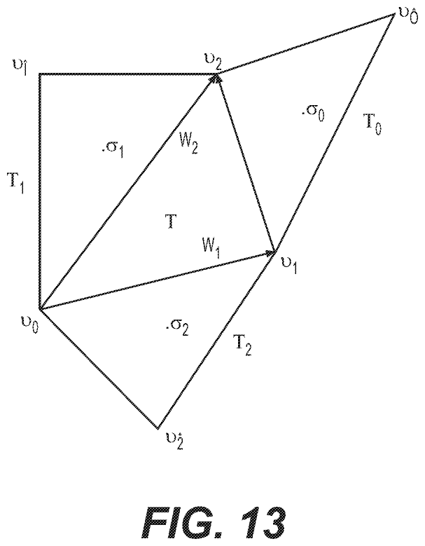

The process of accommodating an implant within breast tissue may be implemented by the following procedure. According to the above, the placement coordinates and volume of the implant have been determined. One consideration illustrated by the two-dimensional example of FIG. 12 is whether the implant ‘tetrahedra’ may overlap with those of the breast volume.

In at least one embodiment, the implant insertion may be performed in two stages, with energy minimization in each. In the first stage, a rigid implant with predetermined shape may be inserted incrementally. In some examples of the methods herein, gravity is not included in this first stage. The rigid boundary may include a small modification to the Gradient Descent when acceleration methods such as the Momentum method are used. At the start of each iteration, surface vertices may be checked for collision with the rigid boundary. Colliding vertices may be moved out to the nearest surface point on the rigid boundary. If a colliding vertex is marked as ‘fixed’, its status may be changed to ‘free’. This allows the vertex to slide tangential to the boundary as needed during the simulation.

After moving the colliding vertices, the energy gradient dE[v] may be computed at every vertex v. In order to limit the tendency of the colliding vertices to move back across the boundary, the component of dE[v] of colliding vertices that is normal to the boundary may be set to zero. The incremental step for each vertex may then be computed using this modified dE and may be applied to the vertex positions as usual. Because colliding vertices may be allowed to move tangentially to the boundary, it is possible that they will cross into the boundary in this step. Accordingly, the first step in which the colliders are returned to the boundary surface may be applied in every iteration.

The boundary itself may be moved gradually towards its final position, allowing the simulation to adjust to the restoration of colliding points to the boundary at each iteration. The second approach may be used with at least some embodiments discussed herein. The implant boundary may initially be positioned back (perpendicular to the chest wall) so that it is just outside the breast tissue model. The model may then be moved incrementally forward at each iteration until it reaches its final position. The simulation may continue for additional iterations to allow the breast tissue model to reach equilibrium.

A global movement of the breast tetrahedra may be thus performed to accommodate the inserted volume, followed by energy minimization to attain equilibrium. In this manner, the rigid implant may be fully inserted.

The second stage may involve allowing for the implant to be flexible and endowed with its own elastic properties. This may be performed by determining the interpenetration forces at and near the interface and moving the interface so that the forces arising from the interpenetration are equal and opposite. The interior points may then be equilibrated. The procedure may then alternate relaxation of the interface and interior points until equilibrium is reached.

In greater detail, the method may proceed as follows. The two-body deformation algorithm may assume that a start with two tetrahedralized volume models, and that a subset of each of the two volumes' surfaces are in contact. In our implementation, the sets of contacting surface vertices of each model may be supplied as input (since they may be known from the previous Implant Insertion step). These vertices could alternatively be computed from the data, however. It may be assumed that the two models are within a predetermined measure of an equilibrium state to begin with. Accordingly, it may be assumed that the two surfaces do not need to ‘slide’ (move tangentially to each other). The surfaces may deform significantly, but they might deform together, without pulling apart, interpenetrating, or sliding.

Let the two tetrahedral models be TM1 and TM2, and their sets of contacting surface vertices be CV1 and CV2. For each vertex v in CV1, the nearest surface triangle (i.e., a face of a tetrahedron at the surface) t in TM2 may be determined, as might be the barycentric coordinates of v with respect to t. (Methods for computing and storing barycentric mappings may be already built into DSD class VertexMap, which may be used for mapping the multi-resolution volumes, and for mapping the scanned surface back to the tetrahedral model.) The mapping may be computed once, before energy minimization.

In a deformation iteration, an energy gradient may be computed at every vertex in the model, and each vertex may be moved a small step (of a predetermined size) in the direction of decreasing energy. In the two-body deformation, the non-contact vertices may still be handled in this way. The contact vertices in CV1 may still have their energy gradients computed. But they may move by interpolating their new positions from the vertices of CV2, using the stored barycentric coordinate mapping. Thus, the vertices in CV1 may always remain at the surface of CV2.

It may be advantageous to address the energy gradients (forces) at the vertices of CV1. These forces may push on the surface of CV2. The barycentric mapping from CV1 vertices to the vertices of CV2 may already be available, which details how to apportion the force from each CV1 vertex to the nearest set of three vertices in CV2. These forces pushed from CV1 to CV2 may be added to the CV2 energy gradients from their own tetrahedral model TM2. The force of TM1 pushing on TM2 may thus be accounted for in the deformation of points in TM2. After the iterative step of the vertices in CV2, the vertices of CV1 may be moved by interpolating their positions from the new CV2 positions.

An embodiment of this algorithm may be summarized as follows:
Input: TM1, CV1, TM2, CV2
Initialization: A mapping M[v]=(t, b0, b1, b2) may be computed from each vertex v in CV1 to the surface points in CV2. In this mapping, t may be the surface triangle of TM2 containing or nearest to v. (b0, b1, b2) may be the barycentric coordinates of v with respect to t. That is, if u0, u1, and u2 are the vertices of triangle t, then v=b0*u0+b1*u1+b2*u2. Note that when using barycentric coordinates, b2=1−b0−b1.
Iteration: The energy gradient dE[v] may be computed at each vertex v in TM1 and TM2. For each vertex v in CV1, the energy gradient may be distributed to its mapped vertices u0, u1, and u2 in CV2: dE[ui]+=dE[v]*bi, i∈{0,1,2}.
The maximum energy gradient dEmax may be computed among all vertices (excluding CV1).
All non-colliding vertices v in TM1 (i.e., not in CV1) may be stepped: v+=dE[v]*(stepsize/dEmax).
All vertices u in TM2 may be stepped: u+=dE[u]*(stepsize/dEmax).

Material Properties

Properties may be specified in terms of the energy held by an entity when in a particular state. In the long term this state may cover position and momentum. In the following discussion only quasi-static equilibria is computed, so consider only position data. In some examples, viscous forces are not addressed, e.g., since they might affect only the transients by which a minimum-energy configuration C is reached, not C itself. There may be two aspects to giving the energy of an elastic entity such as a triangle. One may be the energy as a function of its deformation from a reference or ‘relaxed’ configuration, with minimum energy (without loss of generality, taken as zero), and the other may be specifying that configuration itself. In some cases, the reference state may be taken as the measured initial configuration. The discussion below exemplifies one set of embodiments where the tissue and implant properties may be taken to be isotropic: the generalization to anisotropic elasticity are performed by methods known to those skilled in the art.

Regarding boundary surfaces, this mesh may be fixed, such that position-dependent energies need not be specified.

Material Properties—Skin

The above section “Geometry—Skin” describes the reference geometry of the skin, and outlines how it may be obtained. Three distinct forms of deformation energy are mean bending, tangential strain, and Gaussian curvature elastic energies. In the mesh version, these may live on different-dimensional elements of the mesh. The unit normal Nₗ^{s} of the skin triangles Tₗ^{s} may be provided, as embedded by the current ƒ. These may be computed as the normalized cross-product of any two of the edge vectors of the triangle's image under F. Using the notation of FIG. 10, its normal may be described by equation (38):

$$N = \frac{{W}_{1} \times {W}_{2}}{‖ {W}_{1} \times {W}_{2} ‖} \qquad ( 38 )$$

where the indices that label the particular triangle in question have been omitted. By consistently orienting the lists in the data structure, they may be arranged to consistently point outward. This section may omit the superscript s which stands for skin.

Skin—Mean Bending

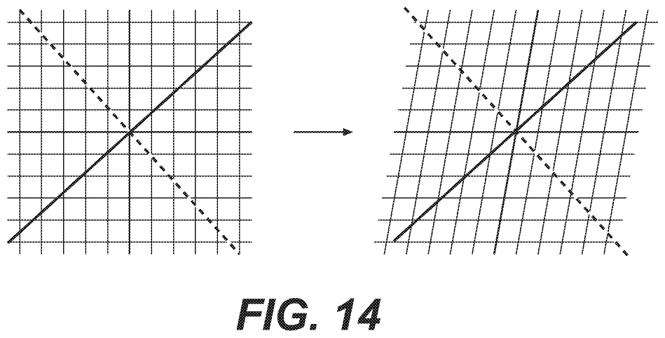

Mean bending is a property that is relative to the ƒ positions of adjacent triangles, for example as shown in FIG. 13. Denoting the mean curvature at a point by H, one form of surface bending is a change of H, as with paper rolled into a cylinder. In at least one embodiment, where each triangle remains flat, such bending may occur along the edges between triangles, though in a smoother description it may be a per-area effect. Skin may have very little resistance to this bending, so it may be neglected in some cases, e.g., in the initial model.

There may be a generalization of the integral of H2 over the areas of triangles sharing an edge. For each edge of length L, sharing faces of areas A1, A2, with A:=½(A1+A2), this energy ∫H²dS is, per edge,

$$3 \frac{{L}^{2}}{A} {sin}^{2} ( θ / 2 ) \qquad ( 39 )$$

where θ=π−α, and α is the (dihedral) angle between the two neighboring triangles at that edge. (If there is little bending the dihedral angle approaches π, so θ→0.) It may be the case that the reference state with respect to which the energy is computed has itself a mean curvature, e.g., such that a discrete expression for ∫(H−H_{ref})²dS may be used, the integration being over the faces sharing the edge. A simplification may be used. To second order, θ/2 may be proportional to ∥N1×N2∥, and moreover, a reference angle may be subtracted from this, using δα=∥N1 N2∥−∥n1×n2∥. The energy increases with bending either way, such that it may be taken to be μ(δα)2 for some μ fixed for each edge. The value of μ may depend on the material, and perhaps on the direction of the edge. For example, an edge aligned with the natural orientation of collagen fibers in the dermis may bend more easily than an edge across them, which may include bending of the individual fibers. If this resistance to bending as localized in the edge were imagined as a kind of angular spring holding stiff triangles, the energy may be designated as proportional to edge length. But since it may be a localized representation of bending that occurs in the triangles themselves, it may be made proportional to the sum of the areas of the triangles that meet at this edge. This area has already been computed, as the denominator in equation (38). A sum over all edges may be determined, and so the proportionality coefficient (elastic modulus) may have dimensions of energy per unit area.

Gaussian Curvature Strain Energy

The Gaussian curvature strain energy at vertex i may be described by equation (48):

E_{Gauss}ⁱ=γ_{Gauss}|Σᵢ^{ref}−Σᵢ^{ƒ}| (48)

where Σᵢ^{ref} and Σᵢ^{ƒ} are defined in the Gaussian curvature section above. The coefficient γ_{Gauss} is related to γ_{length}, since flattening a boss may involve radical contraction and/or circumferential extension. It may be treated as to be empirically estimated. Unlike the per-unit-area tangential strain energy (additive over sub-triangles), the angles at a vertex introduced in a simple mesh subdivision may sum to a constant 2π for both the reference geometry and the embedded geometry, so there might be no weighting of E_{Gauss}ⁱ by the size of the surrounding triangles. The term Σᵢ^{ref} may be computed in the setup stage and stored for repeated use.

Implant Shell/Membrane

With respect to a breast implant model used in some methods discussed herein, the implant shell or membrane may have analogous properties to skin. Thus, energy may be associated with its tangential, mean bending and Gaussian strain, possibly using the same formalism as for skin (though perhaps not the same coefficients).

Tetrahedral Energies

According to some aspects of the methods herein, the gradient directions for the energies associated with the tetrahedra may first be determined, confining the method to isotropic energies to begin with. For example, let Vₗ^{t} be the volume of the initial oriented tetrahedron Tₗ^{t} (see the Breast Volume section above). For each tetrahedron, there may be three energy terms in the isotropic case, described by equations (49)-(51):

E₁=λ₁(Tr[Tₛₜᵣₐᵢₙ])²|Vₗ^{t}|=:λ₁|Vₗ^{t}|e₁ (49)

E₂=λ₂Tr[Tₛₜᵣₐᵢₙ²]|Vₗ^{t}|=:λ₂|Vₗ^{t}|e₂ (50)

E₃=gM_{L}^{ft}Z_{c}=ρgZ_{c}|Vₗ^{t}| (51)

where Mₗ^{ƒt} may be the invariant mass of the tetrahedron in question (hence Mₗ^{ƒt}=Mₗ^{t}), and Z_{c} may be the vertical height of the centroid of the current tetrahedron in a frame oriented with the earth's gravity. The first two are elastic strain energies (e.g., associated with a strain matrix), and the third is the gravitational potential energy. Gravitational potential energy may account for natural movement of breast tissue and/or a breast implant due to gravity as the subject naturally moves (e.g., standing up, walking, lying down, etc.). Thus, for example, the method may include determining a gravitational potential energy of the breast volume model by determining a gravitational potential energy of each tetrahedron. Further, the total gravitational potential energy of the breast volume model may be determined as the sum of the gravitational potential energy of the plurality of tetrahedra. The gravitational potential energy may determined based on the weight, volume, and/or a vertical position of the center of gravity of each tetrahedron of the plurality of tetrahedra. It may be determined that

Z_{c}=¼(sum of heights of vertices) (52)

but since techniques discussed herein have so far expressed the vertex positions in terms of local coordinate systems, explicit expressions for Z_{c} may be postponed. It may also be defined as follows according to equations (53) and (54):

$$\begin{matrix}{e}_{shear} := {Tr [ {T}_{strain} - I Tr ( {T}_{strain} ) / 3 ]}^{2} & ( 53 ) \\ = {e}_{2} - {e}_{1} / 3 & ( 54 )\end{matrix}$$

The e's in equations (53) and (54) may be dimensionless while the E's in equations (49)-(51) may be energies (for each tetrahedron). The gradients of each of these terms with respect to the movement of an arbitrary one of the four vertices Vμ of the tetrahedron in question may be computed. Since a particular vertex may be shared among several tetrahedra, the gradient terms corresponding to that vertex may be added, one for each of the tetrahedra in question. The superscript I of the tetrahedron may be omitted in some cases. The total energy may be the sum of the three energies just stated, over all tetrahedra. The terms λ1 and a λ2 may be moduli of elasticity, while ρ may represent the density of the material in the tetrahedron, and g may be the magnitude of the acceleration due to gravity. Each tetrahedron may have its own elastic modulus and density in general (for sufficiently heterogeneous materials), though as stated below, techniques herein may assume isotropic elasticity properties. The rest of this section may be confined to one tetrahedron since all the energies may be added (and hence all the gradients). However, if any particular vertex is selected to move there may be only a finite number of tetrahedra that will have that vertex, but they all may be considered for the move of that vertex. A sequential algorithm may work with one tetrahedron after another, since a vertex eventually sees all the tetrahedra it is a part of. On the other hand, a parallel algorithm may include the adjacency structure for each vertex. For example, with each vertex, all the tetrahedra which share that vertex may be determined. Variables may be defined according to equations (55) and (56) as:

$$\begin{matrix}{e}_{0} := {( \frac{{V}_{l}^{ft}}{k {V}_{l}^{t}} - 1 )}^{2} & ( 55 ) \\ {E}_{0} := {K}^{tissue} \vert {V}_{l}^{t} \vert {e}_{0} : & ( 56 )\end{matrix}$$

where Vₗ^{t} is the volume of the initial oriented tetrahedron Tₗ^{t} and Vₗ^{ƒt} is the volume of the tetrahedron ƒ(Vₗ^{t}), which may be the image under ƒ of the tetrahedron Tₗ^{t}, also oriented, consistent with the orientation of the vertices in the initial (or in the reference) tetrahedron. In other words, both volumes may be either positive or negative, though a sign change may indicate a tetrahedron that has inverted. To express the fact that the tissue is under blood pressure held in by skin, an equilibrium value of each initial tetrahedron may be estimated as kVₗ^{t}, for some constant k greater than 1, so that the denominator within the parentheses may be a reference tetrahedron, e.g., expanded with respect to the initial. This bulk modulus of the tissue (but see further below) may be described by equation (57):

K^{tissue}=λ+2μ/3 (57)

However, this may introduce a redundancy in the description, both e₀ and e1, for example. However, the virtue of e₀ is that, while θ=+1 it is redundant with one of the other energy terms (albeit taking into account a higher order in the strains), it may also be a penalty term for preventing inversion of the tetrahedra. Therefore, a total energy may be described by equation (58)

Eₜₒₜ=E₀+Geₛₕₑₐᵣ|Vₗ^{t}| (58)

where K in equation (57) and G in equation (58) are the bulk and shear modulus, respectively, of each tetrahedron. Each of these terms may be summed over all the tetrahedra in question. It should be understood that the elastic moduli such as G, K may be effective moduli, so that they need not be related to the same quantities measured for at least a portion of breast tissue or muscle. These moduli may represent the aggregated or properly averaged effects of all the constituents of the breast including muscle, fat, ligaments, etc.

The modified three-dimensional model may reflect an expansion or stretching of tissue, e.g., to accommodate placement of an implant and/or fat grafting. In some examples herein, determining the modified three-dimensional model may include expanding breast tissue of the breast volume model. Thus, for example, the method may include expanding volumes of one or more reference tetrahedra of the plurality of reference tetrahedra associated with the breast volume model. Optionally, the expanding may be based upon a simulation of a surgical injection of fat grafting, e.g., during an implantation procedure or other surgical procedure. In such cases, minimizing the total energy of the breast volume model may be at least partially based upon the expanded volumes of the one or more reference tetrahedra.

According to some aspects of the present disclosure, the total potential energy of the breast volume model may be determined, e.g., by adding the total elastic potential energy and the total gravitational potential energy of the breast volume model. Further, the resting equilibrium position of the breast volume model may be determined, e.g., by minimizing the total potential energy. In some example, the method includes determining a direction that reduces energy, to move each tetrahedron of the plurality of tetrahedra of the breast volume model, to a minimum.

As mentioned above, determining the modified three-dimensional model may include adjusting parameters of a breast surface model. For example, the elastic potential energy of each triangle of a plurality of triangles of the breast surface model may be determined by: (a) determining a two-dimensional strain energy by determining a sum of the plurality of triangles of the breast surface model; (b) determining a bending energy based on an angle of bending of a face of each triangle with respect to faces of neighboring triangles, and/or (c) determining a curvature energy based on an angle deficit of an angle sum at each vertex of the plurality of triangles of the breast surface model.

Further, the method optionally may include simulating movement of one or both breasts and/or the torso of the subject, e.g., to evaluate the natural response of the subject's tissue as the subject moves. This simulation may include determining a time-trajectory of movement of one or more fiducial points associated with the modified three-dimensional model, and determining a plurality of equilibrium positions of the breast(s) of the subject during motion at predetermined time points. Further, at least one three-dimensional time-dependent configuration of the breast(s) and/or the torso based on the determined plurality of equilibrium positions may be displayed, e.g., in real time.

Gradients

Since the triangular and tetrahedral energies may be expressed in terms of the vectors that form the sides of the respective simplex, denoted by W's above, the gradients with respect to the W's may be calculated. Then, the gradients with respect to the vertex positions may be obtained by the chain rule of differentiations. Thus the (sparse) matrix of gradient entries for the entire complex of tetrahedra and triangles may be built up.

The introduction of anisotropic elasticity to the tetrahedra may also be performed.

Combined Strain Energy

In the above, the surface strain and bending energies are described separately. Total pointwise energy Q in some cases may be considered to be the sum of tangential and bending terms, though some cross-terms in its expansion may be ruled out on symmetry grounds. (For example, the product of the ‘mean extension’ (t₁₁+t₂₂)/2 and mean curvature may change sign under reflection and under reversal of the arbitrary orientation used for S, so this term might not belong in the quadratic part of Q—though its square might give a legitimate quartic contribution.) One possibility is to use additive combinations, while designing stress/strain modules where more elaborate Q can easily be slotted in.

For a thin shell of rubber or other elastomer of a breast implant, it may make sense defining Q to go to continuum mechanics, and derive its form and its coefficients by the standard approach to the limit of thinness. For biological surfaces such as skin and breast tissue, this may be less clear. Initially the choice of Q may be heuristic and phenomenological. Refinement of its form, and the coefficient values involved, may be done by system identification and parameter estimation techniques from measurement on larger pieces, as by attempting to identify elastic constants for continuum blocks within the membrane: so small that they only very approximately exist, might not be excised for experiment, and might interact non-uniformly across their boundaries, where bonds happen to lie. Even in more macroscopic causes, such as skin, biological surfaces may have a more complex internal structure than is ever assumed in classical theory. Exemplary techniques presented herein relate energy directly to the vertex positions, so that the time forces on a vertex may be summed and used to move it, at faster speeds than other possible techniques.

Mastopexy and Surgical Simulation Procedures

The method described above may comprise computing the strain of a tetrahedron in its current state situated in three-dimensional space contiguous with other tetrahedra that collectively together define the breast or breast with implant volume model. The method may compute the strain of the tetrahedron with respect to a reference tetrahedron that is defined by other means, such as described above for a gravity-free state. The reference tetrahedron may be defined individually and might not have to be contiguous with any other reference tetrahedron. This allows computational methods to be readily supplied for surgical procedures such as increasing the volume of tissue, e.g., by grafting, or volume reduction, e.g., as in mastopexy procedures. In the first case of volume increase, the method may define a set of reference tetrahedra of increased volume (expanding volumes of one or more reference tetrahedra of the plurality of reference tetrahedra associated with the breast volume model), the reference tetrahedra being chosen according the planned region of volume increase. Similarly, for volume reduction of breast tissue, one may define a set of reference tetrahedra that are reduced in volume, including in some cases a substantial reduction in volume, to allow for the removal of tissue. For example, determining a volume reduction of breast tissue may be performed by reducing volumes of one or more reference tetrahedra of the plurality of reference tetrahedra of the breast volume model.

Similar considerations may apply to procedures where skin flaps are removed and resutured. A set of reference triangles may be defined to have very small area to allow for the surrounding skin triangles to be resutured with tension. Thus, for example, determining a reduction in breast skin surface area may be performed by determining a reduction in area of one or more reference triangles of the plurality of reference triangles of the breast surface model. The reduction in area may be determined, at least in part, based upon a simulated removal of skin and/or resuturing associated with a medical procedure. Further, simulating the reducing in breast skin surface area may include monitoring, e.g., by collision detection, the plurality of tetrahedra of the breast volume model to determine that each tetrahedron of the plurality of tetrahedra remains within a surface of the breast surface model. Further, a resting equilibrium position of the breast surface model may be determined by minimizing a total energy of the breast surface model and breast volume model, e.g., according to a predetermined constraint associated with an integrity of breast tissue. In some aspects herein, the constraint may be based on profile data specific to the subject or may be based on aggregated data, e.g., for subjects similar in physicality.

Numerical Methods for Energy Minimization

In order to obtain information on the breast tissue after deformation or surgery, the position ƒ may need to be determined, as in the triangle geometry section, such that the mesh of the embedded triangles is at an energy minimum, where the energy E is the sum of all the energies defined above. For this purpose, at least one embodiment may use gradient descent or related methods such as Nesterov acceleration, momentum methods, etc. In both cases, the initial configuration ƒₒ obtained (the set of ƒₒv) may be used, and a succession of ƒᵢ may be determined such that energy decreases.

Gradient descent may involve choosing ƒᵢ₊₁ such that the vector {ƒᵢ₊₁v−ƒiv}, curly braces denoting the collection of the individual vectors in three dimensions, may be a particular multiple of the gradient ∇E with respect to the non-fixed vertex positions. Newton's method may linearize the problem of finding ƒᵢ₊₁ such that ∇E(ƒᵢ₊₁)=0 (this linearization may involve the first and second derivatives of E at ƒ, which are here computable analytically), which allows larger steps. For a quadratic problem it may converge in a single step, rather than track gradient curves. Although the energies might not be quadratic in these vectors, it is unlikely that local minima are an issue.

These steps may be summarized as:
1. Obtain input scans and surface triangulations.
2. Compute surface triangulation to obtain closed surfaces followed by tissue volume tetrahedralization and.
3. Describe application-specific procedures (e.g. breast implant simulation).
4. Re-tetrahedralize tissue volume, if necessary.
5. Compute the shrunk reference triangles as needed.
6. Evaluate volumetric and tangential strain, bending, volumetric strain, and curvature energies, and at least their first derivatives with respect to ƒ.
7. Use gradient descent or other method (see below), converging to equilibrium ƒ.
8. Output new tetrahedra and surface triangles and textures.

Gradient Descent

According to some aspects of the present disclosure, minimizing the total energy of the breast volume model uses a gradient descent technique. For example, minimizing the total energy of the breast volume model may use a gradient descent technique that is accelerated by a momentum method, Nesterov accelerations, and/or by interpolating, smoothing, and/or approximating computed movements to allow movement of a plurality of tetrahedra of the breast volume model and/or a plurality of triangles of a breast surface model. Such movements may be of an energy less than a predetermined total energy. A gradient descent algorithm might proceed by moving along the gradient of the energy. In the following, the tetrahedral and the triangular energies may be treated separately for the following reason. The data structures employed during the software implementation may be organized tetrahedrally for the tetrahedra, but for the triangles the adjacency of each vertex to the triangles it belongs to may be utilized. In other words, the data structure of each tetrahedron may contain the vertices and edges that belong to it, while a vertex does not contain the tetrahedra that belong to it. On the other hand, the adjacency relations may go both ways for the triangles. This may mean that the move for a vertex in the direction of steepest descent for the strain energy may be considered incomplete immediately after a vertex has been encountered, since the same vertex may re-occur for a different tetrahedron. The move for any vertex may be complete, for example, only after all the tetrahedra have been examined. Put another way, the outer loop may move through the tetrahedra, the inner one through the vertices.

In the case of triangles, the order of the loops may be reversed. However, below the method is written in two parts. For the triangular strain energies, the same order as for the three-dimensional structures has been retained, namely that the outer loop may move through the triangles. In some cases for the bending energies, this may be reversed.

Gradient descent may have certain considerations arising from local minima, though in several applications, the most notable being “deep learning,” these don't seem to cause much problem. If it turns out that gradient descent is not appropriate, then the method of nonlinear conjugate gradients may be utilized. Briefly, it involves, at each step beyond the first one, (i) a move in a direction which is a linear combination of the steepest descent (or the negative gradient) direction computed as above, and the previous move; and (ii) the step length in the steepest descent direction is not arbitrary but is computed by minimizing the energy along a line given by the steepest descent direction.

Other methods to improve the convergence of the simulation procedures, such as Nesterov acceleration or momentum methods, have been discussed above.

Methods to improve the convergence of the simulation procedures may further include smoothing and averaging procedures for the movement of the tetrahedral and triangle nodes so that a larger number of nodes are involved in movements, but the choice of the modal displacements may be ones that are of low overall energy. Such procedures include may include Parzen estimation, radial (and other) basis functions, choice of low-energy modes by off-line computations of the corresponding eigenvectors of the stiffness matrix, etc. Thus, in the procedures of implant insertion described herein it may not only be the interface nodes that are moved to accommodate the interpenetration forces, but those within a tubular neighborhood of the interface, with a smooth fall off of the forces with distance.

As mentioned above, the methods herein may include use of a breast implant model optionally selected by the subject for performing one or more simulations. According to some aspects of the present disclosure, the method may include determining a total potential energy of the breast volume model and the breast implant model; and determining the resting equilibrium position of the breast volume model and breast implant model, e.g., by minimizing the total potential energy of breast volume model and breast implant model. In some examples, determining the resting equilibrium position of the breast implant model comprises positioning the breast implant model against a chest wall model. Additionally, for example, the method may include determining a plurality of triangles associated with a chest wall model; and marking the plurality of triangles associated with the chest wall model as fixed.

Any of the three-dimensional models and/or modified three-dimensional models disclosed herein may be displayed, e.g., before, during, and/or after a simulation. Exemplary displays (also referred to herein as display devices) include, e.g., computer monitors, television screens, projectors, tablet devices, smartphones, etc.

Exemplary Simulation

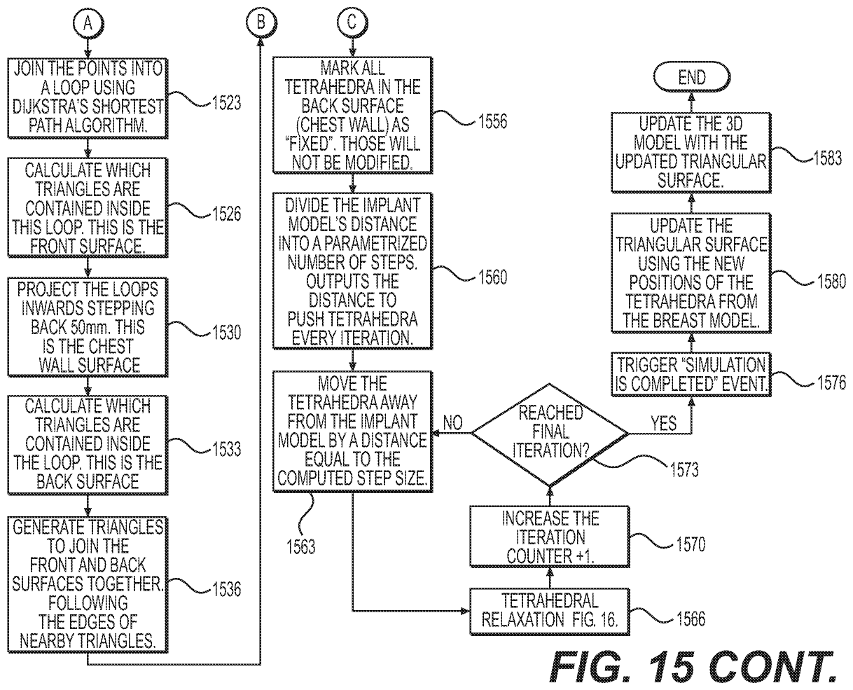

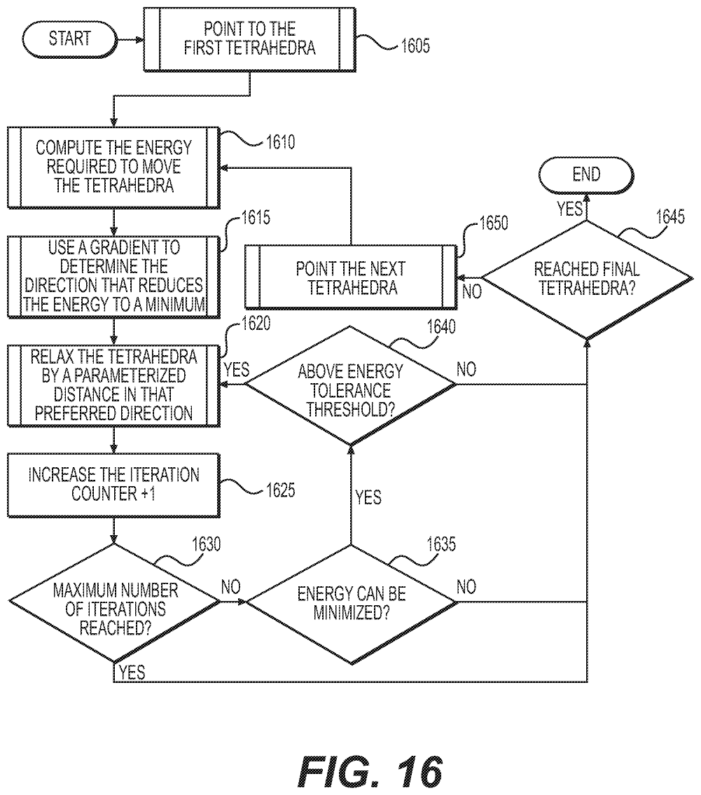

The techniques discussed above will now be discussed in an example implementation that simulates a breast implant in a patient. FIGS. 15 and 16 are flowcharts illustrating a non-limiting example embodiment of the methods herein. In additional examples, various steps of the method may be omitted and/or additional steps may be performed, in accordance with the principles disclosed herein.

In the example of FIGS. 15 and 16, a three-dimensional (3D) model of a human torso may be utilized. It should be understood that individual steps in FIGS. 15 and 16 are exemplary, and may be optional. In addition, alternative or additional steps may be performed, in accordance with techniques discussed elsewhere herein. In step 1503, the 3D model may be imported from a file, such as an OBJ file. The OBJ file may generated by and/or derived from images obtained by the scanner 10. Any other suitable imaging device or imaging system may be used to generate a 3D model used in the methods herein. A tetrahedral model, referred to as a breast volume model, may be generated based on the 3D model.

In particular, at step 1506 it may be determined if the 3D model is facing backwards. If it is, at step 1510, horizontal and depth vertices (x, z) may be inverted. If the 3D model is not facing backwards, at step 1513, the vertices may be scaled, for example by a factor of 1000 to scale from meters to millimeters.

At step 1516, a point of maximum projection for both positive and negative vertices (left and right side of the 3D model) may be determined. A plurality of points, for example eight (8) points, may be marked at a predefined distance around the points of maximum projection determined in step 1513. The points may be joined into a loop, for example using Dijkstra's shortest path algorithm, at step 1520. At step 1526, it may be determined which triangles are contained inside the loop determined in step 1523 (which may be the front surface). To determine the chest wall surface, at step 1530 the loop determined in step 1523 may be projected inwards stepping back a predetermined distance, for example 50 mm. This projection may represent the chest wall surface.

At step 1533, it may be determined which triangles are contained inside the loop determined in step 1523. These triangles may represent the back surface. Triangles may then be generated in order to join the front and back surfaces together, per step 1536. This may be done by following the edges of nearby triangles.

The 3D model may include portions of the torso other than the breast. Thus, the method may comprise analyzing the 3D model to determine which portions of the 3D model is a breast, and constructing one or more breast surface models to represent the determined portions. In the example shown in FIG. 15, breast surface models for the front surface, chest wall surface, and back surface are generated from the 3D model. These models, which are also referred to as breast sub-models, may be used to generate the breast volume model, at step 1540. The breast volume model may comprise tetrahedra and may be considered as a container for the tetrahedra.

Each tetrahedron of the breast volume model may be defined by a plurality of four vertices, for example four vertices, along with other properties of the tetrahedron. Each of the vertices may be defined by a position in three-dimensional space (for example, x, y, and z coordinates). When the simulation is performed, each tetrahedron of the breast volume model may be represented as an object (e.g., a variable or data structure) stored in memory. The object may have a set of properties that include the four vertices that define the tetrahedron. Each vertex of each tetrahedron may itself be an object stored in memory having a set of properties that include the position of the vertex in three-dimensional space. The object for the vertex may also have additional properties, such as a property indicated whether the vertex is “fixed.” The property of “fixed” is discussed later below.

At step 1543, the breast volume model may be de-destressed by applying reversed gravity. This process may be performed in accordance with the methodologies discussed herein, and may result in an estimate of a gravity-free state. For example, determining a reversed gravity model may include simulating the effect of reversed gravity with one or more pre-determined moduli of elasticity of tissue, which may be specific to the patient and/or obtained from aggregated reference data, e.g., such as elasticity of tissue data averaged for a plurality of subject similar in physicality. In some examples herein, the reversed gravity model may be compared with three-dimensional image data of the subject one or more of the fiducial positions used during a scan (e.g., prone, supine, and/or erect positions).

At step 1546, a 3D model of an implant may be imported, for example from an OBJ file.

Additionally, at step 1550, a tetrahedral model representing a breast implant, referred to as an implant volume model, is also generated. The implant volume model may be generated based on, for example, selected characteristics of the breast implant. Such characteristics may include size, shape, surface texture, gel elasticity, gel rheology, and/or shell elasticity. The data structure discussed in the preceding paragraph for breast volume model may also be used for the implant volume model. That is, the tetrahedra, and each vertex of each tetrahedron, may be represented by a respective object having properties.

The breast volume model and the implant volume model may each be a tetrahedral model of the form discussed above. That is, in general, the simulation methodologies discussed herein may be applied to simulate the breast volume model and the implant volume model under various conditions. In particular, the simulation techniques described herein may be used to model the placement of a breast implant by computation of equilibrium states of the breast volume model and the implant volume model when subject to conditions representing insertion of the breast implant.

Using the breast volume model and the implant volume model, insertion and placement of the implant may be simulated. This simulation process may include positioning (the model representation of) the implant by pushing it against (the model representation of) the chest wall surface (e.g., step 1553 in FIG. 15); marking tetrahedra in the back surface as “fixed” (e.g., step 1556 in FIG. 1); dividing the implant volume models distance into a parametrized number of steps (e.g., step 1560 in FIG. 15), and moving tetrahedra away from the implant volume model by a distance equal to the computed step size (e.g., as operation 1563 in FIG. 15).

In step 1553 of FIG. 15, the implant volume may be positioned in a way such that the tip (or face) of the implant volume model is touching the back surface (back face) of the breast volume model.

Step 1556 may include going through at least a portion or all vertices of all tetrahedra of the breast volume model and finding (identifying) those vertices that conform the back surface of the breast. The vertices conforming the back surface of the breast may be created when the chest wall surface is defined; therefore, these vertices may be associated with a property that would permit their identification or may be part of a list of vertices included in the breast volume model. Once the vertices conforming the back surface of the breast are identified, they may be set to “fixed.” For example, the object representing the vertices may be set to have a property indicating a fixed vertex (e.g., setting a property “isFixed” to the value “yes”). When the vertex is “fixed,” the position of the vertex might not be updated during tetrahedral relaxation. Tetrahedral relaxation may involve updating positions of tetrahedral vertices by some amount.

When the implant volume model is in place, the implant volume model may tend to move toward the front. As the implant volume model moves, it may push the tetrahedra of the breast volume model that is in the way. Pushing may indicate moving tetrahedra to a position that is already being occupied by other tetrahedra. This pushing process may be simulated over a plurality of iterations. During this pushing process, the vertices of the breast volume model that were fixed in step 1556 may remain in the same position (i.e., their positions are not changed). Therefore, the tetrahedra of the breast volume model whose positions change may be among the tetrahedra of the breast volume model that are not fixed in step 1556.

As noted, the pushing process may be simulated over a plurality of iterations, as shown in steps 1563, 1566, 1570, and 1573. For iterative simulation, the implant volume model's distance may be divided into a parametrized number of steps, as indicated in step 1560. That is, the distance of the implant volume model may be divided into a plurality of smaller distances, which are the sizes of the steps. These smaller distances may, for example, be of the same distance (e.g., the step size is constant). The distance associated with each step is the distance to push tetrahedra of the breast volume model every iteration of the tetrahedral relaxation process.

In step 1563, tetrahedra of the breast volume model may be moved away from the implant volume model by a distance equal to a step size, for each iteration of a tetrahedral relaxation relation. The “moving” of tetrahedra may be in the form of updating the position (e.g., x, y, z coordinates) of the vertices of the tetrahedra being moved.

At step 1566, tetrahedral relaxation may be performed. Tetrahedral relaxation may be performed using the process shown in FIG. 16, and as discussed elsewhere herein.

When tetrahedral relaxation has reached the final iteration, a simulation complete event may be triggered, as shown in step 1576. The triangular surface model may be updated using the new position of the tetrahedra form the breast model, at step 1580. The 3D model may further be updated with the updated triangular surface model, in step 1583. As shown in FIG. 15, the tetrahedral relaxation thereby derives a modified 3D model of the human torso simulating the presence of the breast implant. The modified 3D model may be stored and/or displayed on a screen or other display.

The method may include variables such as: gravitational tissue relaxation factor(s), tissue elasticity, skin elasticity, chest wall asymmetry, surgical procedure implant dimensions, and/or implant position(s). These variables may be applied or incorporated into algorithms of the method during various steps or states of the method.

Processes associated with one breast (or surrounding tissue) may also be performed for the other breast (or surrounding tissue). For example, the generation of the breast volume model may be performed twice (once for each breast), such that two implant volume models may be created (one for each breast) and their placements respectively simulated. Therefore, the modified 3D model generated at the end of the process shown in FIG. 15 may simulate the presence of both implants.

The tetrahedral relaxation techniques of FIG. 16 will now be described. At step 1605, the first tetrahedron may be determined. At step 1610, the energy required to move the tetrahedron may be computed. A gradient may be used to determine the direction that reduces the energy to a minimum, at step 1615. At step 1620, the tetrahedron may be relaxed by a parameterized distance in the selected direction. The iteration counter may be incremented by one, at step 1625.

If the maximum number of iterations has not been reached, which may be determined at step 1630, it may be determined if the energy can be minimized, at step 1635. If the energy can be further minimized, it may be determined if the simulation is above an energy tolerance threshold, at step 1640. If above a predetermined energy tolerance threshold, the solution may iterate back to step 1620. If the maximum number of iterations has been reached, or the energy may not be further minimized, or the simulation is not above an energy tolerance threshold, the algorithm may proceed to step 1645. If the final tetrahedron has not been reached, the algorithm may point to the next tetrahedron, at step 1650, and iterate through the steps again beginning at step 1610. The tetrahedral relaxation steps of FIG. 16 may thus iterate until all tetrahedra have been addressed and possibly relaxed.

While FIG. 16 shows tetrahedral relaxation to simulate movement of tetrahedra away from the implant volume model, the tetrahedral relaxation process shown in FIG. 16 and described herein may be used to simulate other processes. For example, the tetrahedral relation process may be used to simulate the effect of the implant volume model and the breast volume model on each other. The tetrahedral relaxation process may be used in reverse to simulate the removal of an implant, placement of an implant with a smaller volume, or a breast reduction.

Methods for simulating placement of an implant according to the present disclosure, e.g., an example of a breast implant simulation illustrated in FIGS. 15 and 16, may be performed by one or more processors configured to perform the method. The one or more processors may be coupled to, or have access to, a memory storing instructions that, when executed by the one or more processors, cause the one or more processors to perform the method for simulating placement of a breast implant. The one or more processors may, for example, be or include a CPU, a GPU, and/or a plurality of CPUs and/or GPUs. The memory may comprise a non-transitory computer-readable storage medium. In some embodiments, the instructions may be stored on a non-transitory computer-readable storage medium that is independent of any processor or computer system.

According to some embodiments of the present disclosure, the method for simulating placement of a medical implant (e.g., a breast implant) may be performed by the medical imaging system disclosed in WO 2017/175055, incorporated by reference herein. For example, the one or more processors discussed in the preceding paragraph above may be a part of the computer system 90 shown in FIG. 1, and may operate in conjunction with other components of the computer system 90 or the imaging system disclosed in WO 2017/175055.

It is noted that, while FIGS. 15 and 16 illustrate various operations in the format of a flow diagram, the order of the operations are not necessarily limited to the specifically depicted order. Further, other examples of the method may include fewer or more operations than those illustrated in FIGS. 15 and 16.

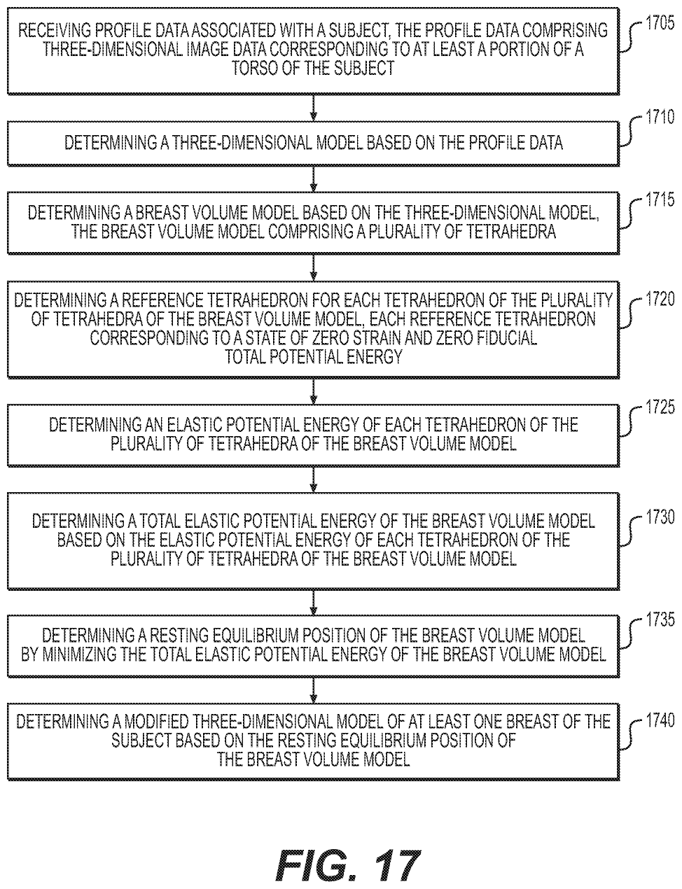

FIG. 17 discloses a flow chart of an exemplary method for processing images to determine a modified image in accordance with techniques presented herein. At step 1705, profile data associated with a subject may be received, the profile data comprising three-dimensional image data corresponding to at least a portion of a torso of the subject. At step 1710, a three-dimensional model may be determined based on the profile data. At step 1715, a breast volume model may be determined based on the three dimensional model, the breast volume model comprising a plurality of tetrahedra. At step 1720, a reference tetrahedron for each tetrahedron of the plurality of tetrahedra of the breast volume model may be determined, each reference tetrahedron corresponding to a state of zero strain and zero fiducial total potential energy. At step 1725, an elastic potential energy of each tetrahedron of the plurality of tetrahedra of the breast volume model may be determined. At step 1730, a total elastic potential energy of the breast volume model may be determined based on the elastic potential energy of each tetrahedron of the plurality of tetrahedra of the breast volume model. For example, the elastic potential energy may correspond to energy due to a strain matrix of each tetrahedron. At step 1735, a resting equilibrium position of the breast volume model may be determined by minimizing the total elastic potential energy of the breast volume model. At step 1740, a modified three-dimensional model of at least one breast of the subject may be determined based on the resting equilibrium position of the breast volume model.

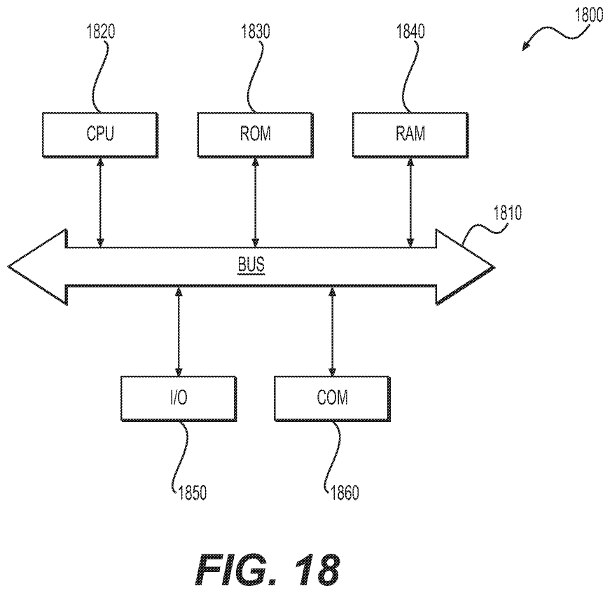

Additional notes on techniques, software, and hardware embodiments will now be discussed. With reference to FIG. 1, the computer system 90 (along with its associated software) may control the scanner 10 while scanning a subject, perform image processing of the image data received from the scanner 10, and/or perform simulations on the resulting images. In the description below, although the computer system 90 will be described as performing these and other functions, as will be understood by people skilled in the art, software algorithms running on the hardware components (e.g., microprocessors, etc.) of the computer system 90 may in fact perform these functions. Further, although computer system 90 in FIG. 1 is illustrated as a single desktop computer, this is only exemplary. In general, computer system 90 may include any type of computing devices (e.g., single-board computers, microcontrollers, general-purpose computers, personal computers, etc.), an example of which is shown in FIG. 18.

FIG. 18 is a simplified functional block diagram of a computer system that may be configured as the scanner 10, controller 70, system 90, and/or server(s) 97, for executing the methods, according to exemplary an embodiment of the present disclosure. Specifically, in one embodiment, any of the user devices, servers, and/or exchanges may be an assembly of hardware, e.g., system 1800, including, for example, a data communication interface 1860 for packet data communication. The platform may also include a central processing unit (“CPU”) 1820, in the form of one or more processors, for executing program instructions. The platform typically includes an internal communication bus 1810, program storage, and data storage for various data files to be processed and/or communicated by the platform such as ROM 1830 and RAM 1840, although the system 1800 often receives programming and data via network communications. The system 1800 also may include input and output ports 1850 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

Examples of computing devices that may be used in the computer system 90 may include, but are not limited to, the Intel® Edison microcontroller, the Arduino microcontroller, and the Intel® Next Unit of Computing (NUC). In some embodiments, the computer system 90 may include multiple electronic devices (computers, servers, smartphones, tablets, personal digital assistants (PDAs), etc.) in wired or wireless communication with each other. For example, in some embodiments, computer system 90 may include a computer in direct communication with the controller 70 and multiple other electronic devices (server systems, memory systems storing databases, smartphones, PDAs, etc.) wirelessly coupled to the computer over the internet or other known communication network 95 (LAN, PLAN, etc.). In some embodiments, computer system 90 may include multiple computational devices configured to perform different specialized functions. For instance, these multiple devices may include a first computer configured as a microcontroller that controls the sensors, actuators, motors, and other scan related systems of the scanner 10, and a second computer that controls the image processing and management (saving, cataloging, retrieving, etc.) aspects of the system. The first computer may include components such as analog digital converters (ADC) and pulse width modulation (PWM) components. In some embodiments, the first computer may include software modules configured to optimize the scanning capabilities of the scanner 10 for different portions of the body (such as, e.g., the torso, etc.), and the second computer may include software modules configured to optimize the image processing and data management capabilities. The first computer may communicate with the second computer (e.g., a PC) through one or more communications ports. In some embodiments, the first computer and the second computer may be separate components, while in other embodiments, the first and second computers may be parts of the same computer system.

The computer system 90 may include associated input devices (e.g., keyboard, mouse, touchscreen, etc.) that enables a user (doctor, technician, etc.) to provide input to the computer system 90. Using these input devices, the user may input relevant information of a new subject (name, address, height, weight, dimensions, and other relevant information) into the computer system 90. This information may be stored in a database associated with the computer system 90 (i.e., locally or remotely located) as the subject's profile. The profile of a subject may include any information identifying the subject (e.g., the subject's first name, last name, date of birth) and the type of scan (e.g., torso, facial, other). In some embodiments, the profile may include information on the subject's medical history (e.g., prior medical procedures, medicines taken, etc.), and/or information about any medical implants that the subject may have. For example, the subject profile may indicate whether the subject has any breast implants or other implants or prosthetics, the type of each implant and its location, the implant manufacturer, date of manufacture, warranty information, and/or serial number of each implant. Additionally or alternatively, the patient profile may include medical data such as blood pressure and the existence or absence of any allergies or other medical conditions. The user may view (and modify if needed) the profiles stored in the database. While scanning a preexisting subject (i.e., a subject for whom a profile has already been created), the user may select the subject's profile from the database.

The computer system 90 may include image scan routines (or scanning algorithms) that, when activated, direct instructions to the components of the scanner 10 (imaging device, cart, LEDs, etc.) to perform a scan. These scan routines may include software modules written using any type of computer language. In some embodiments, the scan routines may include one or more application programming interface (API). Computer system 90 may also include software algorithms or modules configured for processing the image data received from the scanner 10, computation modules configured to extract desired features (dimensions, etc.) from the images, and one or more simulation modules configured to perform the desired simulations (explained further herein). Each of these software modules may generate one or more include graphical user interfaces (GUIs) or windows on the display device 92 that enable the user to provide input to the computer system 90. Although the software modules that control scanning, image processing, and simulation are described as being included on the computer system 90, this is only exemplary. In some embodiments, one or more of these modules may be remotely located (e.g., in a cloud server, or on network server(s) 97) that may be accessed by computer system 90. Controller 70 may serve as an interface between the computer system 90 and the components of the scanner 10. For instance, the controller 70 may convert signals between the computer system 90 and the scanner 10 to a form that will be recognized by each component.

In some embodiments, the controller 70 may control the movement of the imaging device (translations and/or rotations in the x, y, and z axis) based on input from the sensors (e.g., sensors) of the scanner 10. The controller 70 may be configured to control horizontal and vertical translations (and/or rotations) of imaging device simultaneously or in a serial manner. For example, obtaining an image may include moving a cart (e.g., cart 34 of WO 2017/175055) along a rail (e.g., rail 32 of WO 2017/175055) of the imaging device, wherein the moving includes moving the cart to the first end of the rail, and moving the cart from the first end to the second end. The operation of moving the cart to the first end may include rotating one or more cameras (e.g., cameras disclosed in WO 2017/175055) by an angle between about 5-45 degrees, or between about 30-45 degrees, in one or more axes.

In some embodiments, the controller 70 may serve as a standardized hardware component that enables the scanner 10 to operate with different types of computer systems 90 (e.g., smartphone, server system, etc.). In some embodiments, the controller 70 may be custom-made to enhance the capabilities of a specific computer system 90 (e.g., enhance Intel® Edison's capabilities). Although computer system 90 and the controller 70 are described and illustrated as being separate components, this is only exemplary. In some embodiments, the functions of both the computer system 90 and the controller 70 may be integrated into a single component. In some embodiments, communications between the computer system 90, controller 70, and/or the scanner 10 may be through a serial communications link (e.g., using the communication (COMM) ports in a PC's operating system). These serial communications may be provided from the scanner API to the computer system 90 and vice versa.

Although not illustrated in the figures, imaging system 100 may include one or more power supplies configured to provide external electric power to components (scanner 10, controller 70, computer system 90, etc.) of the system. Any type of power supply capable of providing sufficient current and voltage to operate these components may be used. The power supplies may be integral with the components or may be a separate part electrically coupled to the components. Although not a requirement, in some embodiments, an external 24V power supply may be coupled to a peripheral controller, and an external 12V power supply connected to a motor controller, of controller 70. In some embodiments, these external power supplies may be controlled independently, such that they are not connected directly to the controller 70. In some aspects, an external 9V power supply may also be connected to the computer system. In some embodiments, some or all components of the imaging system 100 (e.g., scanner 10, controller 70, computer system 90, etc.) may also include internal power supplies (e.g., one or more batteries) that provide power to these components. The internal power supplies may be used when external power is not available (e.g., blackout, etc.), for example, or is not stable (e.g., voltage fluctuations, etc.). Such internal power supplies may also facilitate transport and/or use of the imaging system 100 in different locations.

The imaging system 100 may be configured to perform any type of scan (torso scan, etc.) or a combination of scans on the subject using the scanner 10. The user may request a scan of the subject via the computer system 90. In some embodiments, the user may manually enter the parameters needed for the scan into the computer system 90 (e.g., into a GUI) using an input device (e.g., keyboard, mouse, touchscreen, etc.), and initiate the scan (e.g., by pressing a key on the keyboard, selecting an icon in the GUI, etc.). In some embodiments, various customized scan routines (e.g., facial scan, torso scan, etc.) may be preprogrammed into the computer system 90, and the user may select one of these customized scan routines to scan a subject. These scan routines may prescribe various parameters for the scanner 10 to use while scanning the subject.

Any variable that affects the image captured by the imaging device during the scan may be a parameter. Such parameters include, for example, the parameters described in WO 2017/175055, incorporated by reference herein. For example, the parameters may include variables that affect the quality of the obtained image, such as, for e.g., color, texture, landmarks, depth, scan type, scanning area (width, height, depth), etc. The parameters may also include the trajectory (e.g., x, y, z translations, etc.) of the imaging device during the scanning, settings for scan speed (e.g., speed of x, y, z translations, etc.), settings for the LEDs (which LEDs to turn on, when the LEDs are turned on, wavelength(s) of light, etc.), settings for the cameras, etc. In some scanning applications, a single scan of a target area of the subject may be sufficient to generate a suitable image, while in other applications, multiple scans may be needed for a suitable image. Therefore, in some embodiments, the scan routine may also define the number of scans to be taken of a particular area, and the variables for use in image processing (e.g., for combining the data obtained from the multiple scans into a single image file).

In some examples herein, the imaging system may be used in combination with a turntable. For example, a turntable may be used to rotate a subject, such that the scanner is able to generate a 360° image of the subject. The turntable may be provided for the seating of the subject. That is, the seating may be placed on a turntable to permit the subject to rotate 360°. Additionally or alternatively, the imaging system may be portable, e.g., such that an operator may move the imaging system or component thereof (such as the scanner of the imaging system or an imaging component of the scanner) around the subject to obtain a 360° image of the subject. In such an example, the rail may be omitted.

The imaging device (of the scanner 10) may acquire a real-time image of the subject positioned in front of the scanner 10 and display the image on the display device 92 of the computer system 90. The user may use this real-time image to adjust the position of the subject in front of the scanner 10 before acquiring images. In some embodiments, the computer system 90 may include features that assist the user in correctly positioning the subject. For example, augmented reality features, such as, lines, grids, or “other indicators may be presented on the display, along with the subject's real-time image, to assist the user in correctly positioning the subject. The location of these indicators on the display device 92 may be based on calculations for reference anatomical features of the subject. For example, based on the information (height, dimensions of features, etc.) in the subject's profile, the location of indicators corresponding to relevant body features (e.g., sternal notch for a torso scan, etc.) may be calculated, and identified on the display device 92. Using the displayed indicators as a guide, the user may adjust the position of the imaging device/scanner 10 (or the subject in front of the imaging device/scanner 10) such that the indicators are positioned at the correct location on the subject's image.

The computer system 90 may include any number of customized scan routines that a user may choose from. For example, in some embodiments, a customized software module (AX3) may be provided in computer system 90. The AX3 software module may be configured to perform a scan (e.g., activate and control the scanner to get images from the scanner) and perform image processing and simulations on the resulting images. For example, the AX3 software module may control the scanner API (subroutine) that performs the scan routines to capture the 3D images. In some embodiments, the 3D images may include three files per image. The AX3 module may convert the 3 files of one image to a single file. In some embodiments, the AX3 module may perform profiling TBS (Tissue Behavioral System) where properties such as skin elasticity, muscle and glandular tissue properties are assigned to sections of the image and results computed. In some embodiments, the AX3 module may also enable the user to select a desired implant from a catalog of implants (Implant Catalogue selection) of one or multiple manufacturers (e.g., catalog of Motiva implants), perform simulations, enable automated anatomical measurement, enable the patient to view the results of the analysis (scanned image and/or the expected result of implanting a selected implant on the body) on the display device 92 of the computer system 90.

In some embodiments, the available scan routines are presented on the display device 92 in a manner that enables the user to select one for application on a subject. The chosen scan routine may be used to image the subject without modification, or the user may modify the parameters associated with a chosen scan routine before initiating the scan. In some embodiments, the computer system 90 may have different APIs for different scan routines. For example, the display device 92 of the computer system 90 may list the available scan routines (e.g., torso scan, facial scan, full body scan, etc.), and allow the user to select the desired scan routine. The computer system 90 may then execute the API associated with the selected scan routine.

While the figures and disclosure herein depict several exemplary configurations of systems, assemblies, implants, and methods, one of ordinary skill in the art will understand that many other configurations and variations on methods are possible and may be appropriate for a given implant, patient, procedure, or application, e.g., based on implant size, shape, orientation and intended location in the patient's body. The examples of devices, systems, and methods herein are intended to be exemplary and are not comprehensive; one of ordinary skill in the art will also understand that some variations on the disclosed devices, systems, and methods herein are also contemplated within this disclosure.

We claim:

1. A computer-implemented method of processing images to determine a modified image, comprising:
receiving profile data associated with a subject, the profile data comprising three-dimensional image data corresponding to at least a portion of a torso of the subject;
determining a three-dimensional model based on the profile data;
determining a breast volume model based on the three dimensional model, the breast volume model comprising a plurality of tetrahedra;
determining a reference tetrahedron for each tetrahedron of the plurality of tetrahedra of the breast volume model, each reference tetrahedron corresponding to a state of zero strain and zero fiducial total potential energy;
determining an elastic potential energy of each tetrahedron of the plurality of tetrahedra of the breast volume model;
determining a total elastic potential energy of the breast volume model based on the elastic potential energy of each tetrahedron of the plurality of tetrahedra of the breast volume model;
determining a resting equilibrium position of the breast volume model by minimizing the total elastic potential energy of the breast volume model; and
determining a modified three-dimensional model of at least one breast of the subject based on the resting equilibrium position of the breast volume model.

2. The computer-implemented method of claim 1, wherein each reference tetrahedron is determined using the three-dimensional image data, wherein the three-dimensional image data includes three-dimensional images of the subject in at least two fiducial positions chosen from a prone position, a supine position, and an erect position.

3. The computer-implemented method of claim 2, wherein determining each reference tetrahedron further comprises:
determining a reversed gravity model simulating an effect of reversed gravity with one or more pre-determined moduli of elasticity of tissue; and
comparing the reversed gravity model with the three-dimensional image data of the subject in one of the at least two fiducial positions.

4. The computer-implemented method of claim 1, wherein the elastic potential energy corresponds to an energy due to a strain matrix of each tetrahedron of the plurality of tetrahedra.

5. The computer-implemented method of claim 4, further comprising:
simulating movement of the at least one breast and/or the torso of the subject by:
determining a time-trajectory of movement of one or more fiducial points associated with the modified three-dimensional model;
determining a plurality of equilibrium positions of the at least one breast of the subject during motion at predetermined time points; and
displaying, in real time, at least one three-dimensional time-dependent configuration of the at least one breast and/or the torso based on the determined plurality of equilibrium positions.

6. The computer-implemented method of claim 5, further comprising:
determining a reduction in breast skin surface area by:
determining a reduction in area of one or more reference triangles of the plurality of reference triangles, the reduction in area being determined, at least in part, based upon a simulated removal of skin and/or resuturing associated with a medical procedure;
monitoring, by collision detection, the plurality of tetrahedra of the breast volume model to determine that each tetrahedron of the plurality of tetrahedra remains within a surface of the breast surface model; and
determining a resting equilibrium position of the breast surface model by minimizing a total energy of the breast surface and volume models according to a predetermined constraint associated with an integrity of breast tissue.

7. The computer-implemented method of claim 1, further comprising:
determining a gravitational potential energy of the breast volume model by determining a gravitational potential energy of each tetrahedron of the plurality of tetrahedra;
determining a total gravitational potential energy of the breast volume model as a sum of the gravitational potential energy of the plurality of tetrahedra;
determining a total potential energy of the breast volume model, the total potential energy being a sum of the total elastic potential energy and the total gravitational potential energy; and
determining the resting equilibrium position of the breast volume model by minimizing the total potential energy.

8. The computer-implemented method of claim 7, wherein the gravitational potential energy is determined based on a weight, a volume, and/or a vertical position of a center of gravity of each tetrahedron of the plurality of tetrahedra.

9. The computer-implemented method of claim 1, further comprising:
determining a breast surface model based on the breast volume model, the breast surface model comprising a plurality of triangles common with corresponding surface triangle faces of the plurality of tetrahedra of the breast volume model; and
determining a reference triangle for each triangle of the plurality of triangles of the breast surface model, each reference triangle corresponding to a state of zero strain and zero fiducial total potential energy.

10. The computer-implemented method of claim 9, further comprising:
determining a plurality of skin tension lines associated with the breast surface model, wherein the reference triangle for each triangle of the plurality of triangles is based on the plurality of skin tension lines.

11. The computer-implemented method of claim 9, further comprising:
determining an elastic potential energy of each triangle of the plurality of triangles of the breast surface model by performing one or more steps comprising:
(a) determining a two-dimensional strain energy by determining a sum of the plurality of triangles of the breast surface model;
(b) determining a bending energy based on an angle of bending of a face of each triangle with respect to faces of neighboring triangles; and/or
(c) determining a curvature energy based on an angle deficit of an angle sum at each vertex of the plurality of triangles of the breast surface model.

12. The computer-implemented method of claim 9, wherein minimizing the total energy of the breast volume model uses a gradient descent technique that is accelerated by a momentum method, Nesterov accelerations, and/or by interpolating, smoothing, and/or approximating computed movements to allow movement of the plurality of tetrahedra of the breast volume model and/or the plurality of triangles of the breast surface model, the movements being of an energy less than a predetermined total energy.

13. The computer-implemented method of claim 1, wherein minimizing the total energy of the breast volume model uses a gradient descent technique.

14. The computer-implemented method of claim 1, further comprising:
expanding breast tissue of the breast volume model by expanding volumes of one or more reference tetrahedra of the plurality of reference tetrahedra associated with the breast volume model, the expanding being based upon a simulation of a surgical injection of fat grafting, wherein minimizing the total energy of the breast volume model is based upon the expanded volumes of the one or more reference tetrahedra.

15. The computer-implemented method of claim 14, further comprising simulating a mastopexy procedure by:
determining a volume reduction of breast tissue by reducing volumes of one or more reference tetrahedra of the plurality of reference tetrahedra of the breast volume model, wherein minimizing the total energy of the breast volume model is based on the plurality of tetrahedra.

16. The computer-implemented method of claim 1, further comprising:
determining a volume reduction of breast tissue by reducing volumes of one or more reference tetrahedra of the plurality of reference tetrahedra of the breast volume model, wherein minimizing the total energy of the breast volume model is based on the plurality of tetrahedra.

17. The computer-implemented method of claim 1, further comprising:
receiving a breast implant model;
determining a total elastic potential energy of the breast implant model; and
determining a resting equilibrium position of the breast implant model by minimizing a total energy of the breast implant model using the total elastic potential energy of the breast implant model.

18. The computer-implemented method of claim 17, further comprising:
determining a total potential energy of the breast volume model and the breast implant model; and
determining the resting equilibrium position of the breast volume model and breast implant model by minimizing the total potential energy of breast volume model and breast implant model.

19. The computer-implemented method of claim 17, wherein the breast implant model is based on one or more implant parameters chosen from implant size, implant shape, implant surface texture, elasticity of a gel filling material, rheology of a gel filling material, elasticity of a shell of the implant, or a combination thereof.

20. The computer-implemented method of claim 17, wherein determining the resting equilibrium position of the breast implant model further comprises positioning the breast implant model against a chest wall model.

21. The computer-implemented method of claim 17, further comprising:
determining a plurality of triangles associated with a chest wall model; and
marking the plurality of triangles associated with the chest wall model as fixed.

22. The computer-implemented method of claim 1, wherein minimizing the total elastic potential energy of the breast volume model comprises:
determining a direction that reduces energy, to move each tetrahedron of the plurality of tetrahedra of the breast volume model, to a minimum.

23. The computer-implemented method of claim 1, further comprising:
displaying the modified three-dimensional model on a display.

24. The computer-implemented method of claim 1, wherein the profile data comprises at least one characteristic of the subject chosen from a gravitational tissue relaxation factor, tissue elasticity, skin elasticity, chest wall asymmetry, or a combination thereof.

25. The computer-implemented method of claim 1, wherein the profile data is stored in a database.

26. A system for processing images to determine a modified image, comprising:
at least one data storage device storing instructions for processing images to determine a modified image; and
at least one processor configured to execute the instructions to perform a method comprising:
receiving profile data associated with a subject, the profile data comprising three-dimensional image data corresponding to at least a portion of a torso of the subject;
determining a three-dimensional model based on the profile data;
determining a breast volume model based on the three dimensional model, the breast volume model comprising a plurality of tetrahedra;
determining a reference tetrahedron for each tetrahedron of the plurality of tetrahedra of the breast volume model, each reference tetrahedron corresponding to a state of zero strain and zero fiducial total potential energy;
determining an elastic potential energy of each tetrahedron of the plurality of tetrahedra of the breast volume model;
determining a total elastic potential energy of the breast volume model based on the elastic potential energy of each tetrahedron of the plurality of tetrahedra of the breast volume model;
determining a resting equilibrium position of the breast volume model by minimizing the total elastic potential energy of the breast volume model; and
determining a modified three-dimensional model of at least one breast of the subject based on the resting equilibrium position of the breast volume model.

27. The system of claim 26, wherein the at least one data storage device comprises one or more databases storing the profile data.

28. The system of claim 26, wherein the system further comprises a display device for displaying the modified three-dimensional model, the display device comprising a screen of a computer, television, projector, tablet device, and/or smartphone.

29. A non-transitory computer-readable medium storing instructions that, when executed by a processor, cause the processor to perform a method of processing images to determine a modified image, the method including:
receiving profile data associated with a subject, the profile data comprising three-dimensional image data corresponding to at least a portion of a torso of the subject;
determining a three-dimensional model based on the profile data;
determining a breast volume model based on the three dimensional model, the breast volume model comprising a plurality of tetrahedra;
determining a reference tetrahedron for each tetrahedron of the plurality of tetrahedra of the breast volume model, each reference tetrahedron corresponding to a state of zero strain and zero fiducial total potential energy;
determining an elastic potential energy of each tetrahedron of the plurality of tetrahedra of the breast volume model;
determining a total elastic potential energy of the breast volume model based on the elastic potential energy of each tetrahedron of the plurality of tetrahedra of the breast volume model;
determining a resting equilibrium position of the breast volume model by minimizing the total elastic potential energy of the breast volume model; and
determining a modified three-dimensional model of at least one breast of the subject based on the resting equilibrium position of the breast volume model.

30. A system for processing images to determine a modified image, comprising:
at least one data storage device storing instructions for processing images to determine a modified image; and
at least one processor configured to execute the instructions to perform a method comprising steps of any of claims 1 through 25.

31. A non-transitory computer-readable medium storing instructions that, when executed by a processor, cause the processor to perform a method of processing images to determine a modified image, the method comprising steps of any of claims 1 through 25.
